SEQUENCE LISTING (44 sequences)

SEQ ID NO: 1 - DNA, 1354 bp, Triticum aestivum
caacatacggcaactcatttgtaatttactgaacagttagtcagctaccatggtgtataagacctgctataccatattgttgtcgtatattcttatataagccttttctcataccatctcatgtatatgaaatctgtcatgcacgcaaacacgcaaccactgagggtacgccacgttgaactagcaggctcttgtttctcaatgcaagcaagctccgttggctgttaggattagatcctaaccaccttaggtgggagagaaggcgccgccgctgtggcatttgtcgtggtcttctagattacctgctcggtcaccttaggggtacctctaggtatttgtttttactttttgagacaaactcatataaagcttttaatttattccaccgtcacaagatttacagggttgaaaacaagatctccgggctgtcctaaaccaagcatggcggtcagtacccagcgttaaagtgtgctttgcaaagaagcacaatgttttgttccaaggatgcgaacatggcaccagccgtctgtctccgcccattctgcatcctcctgctctacttactttacacttacagtacaagcaggtatgtcaccttatttattgaaaatttgacctcatatcactctcaattacaggggctggcgaataataaaacaaactaaaggaaggatatggattatgggaagccatgtgtaagatcaatggagtggagggctaaacaaggaaagtacacttcggatttcgaatcactcccagctaattctgttttctcggggaagcaacctgttctagatttggaatcactcccaaccaatcttcttattggggaaacaatttgtgccagatttggaatcactcccaactaattctgttttccaggggaaacaattaatccacgccacctaaacatgagtaaagtacgaagtacacgtactcaagtctatctcgctatatcggtgggtgttttgtgcgttagtaccatgtaaaaaataaatagatgacaaactgcattgctcgttaacttgcacgctagcactaagtacacctacagagatagatgtagagccctccatatgtggtgccttttacgcttaatgacaatgaaatgcgcgccagctgcattgctttttcacttgtgtgctgcaaatatgtacacgcgaacggatgtagagtattccatatataggtggtggtttgtacgtcaatacttgtctcatatccgccaaatgcatagcttttccactttgttgcgggcaccaaatagacaaatcggactataaattgatacgcccaaggcaaagcccatagaacaagggactacacagacataaagagatccataacttaacct

SEQ ID NO: 2 - DNA, 1354 bp, Triticum aestivum
caacatacggcaactcatttgtaatttactgaacagttagtcagctaccatggtgtataagacctgctataccatattgttgtcgtatattcttatataagccttttctcataccatctcatgtatatgaaatctgtcatgcacgcaaacacgcaaccactgagggtacgccacgttgaactagcaggctcttgtttctcaatgcaagcaagctccgttggctgttaggattagatcctaaccaccttaggtgggagagaaggcgccgccgctgtggcatttgtcgtggtcttctagattacctgctcggtcaccttaggggtacctctaggtatttgtttttactttttgagacaaactcatataaagcttttaatttattccaccgtcacaagatctacagggttgaaaacaagatctccgggctgtcctaaaccaagcatggcggtcagtacccagcgttaaagtgtgctttgcaaagaagcacaatgttttgttccaaggatgcgaacatggcaccagccgtctgtctccgcccattctgcatcctcctgctctacttgctttacacttacagtacaagcaggtatgtcaccttatttattgaaaatttgacctcatatcactctcaattacaggggctggcgaataataaaacaaactaaaggaaggatatggattatgggaagccatgtgtaagatcaatggagtggagggctaaacaaggaaagtacacttcggatttcgaatcactcccagctaattctgttttctcggggaagcaacctgttctagatttggaatcactcccaaccaatcttcttattggggaaacaatttgtgccagatttggaatcactcccaactaattctgttttccaggggaaacaattaatccacgccacctaaacatgagtaaagtacgaagtacacgtactcaagtctatctcgctatatcggtgggtgttttgtgcgttagtaccatgtaaaaaataaatagatgacaaactgcattgctcgttaacttgcacgctagcactaagtacacctacagagatagatgtagagccctccatatgtggtgccttttacgcttaatgacaatgaaatgcgcgccagctgcattgctttttcacttgtgtgctgcaaatatgtacgcgcgaacggatgtagagtattccatatataggtggtggtttgtacgtcaatacttgtctcatatccgccaaatgcatagcttttccactttgttgcgggcaccaaatagacaaatcggactataaattgatacgcccaaggcaaagcccatagaacaagggactacacagacataaagagatccataacttaacct

SEQ ID NO: 3 - DNA, 1353 bp, Triticum aestivum
caacatacggcaactcatttgtaatttactgaacagttagtcagctaccatggtgtataagacctgctataccatattgttgtcgtatattcttatataagccttttctcataccatctcatgtatatgaaatctgtcatgcacgcaaacacgcaaccactgagggtacgccacgttgaactagcaggctcttgtttctcaatgcaagcaagctccgttggctgttaggattagatcctaaccaccttaggtgggagagaaggcgccgccgctgtggcatttgtcgtggtcttctagattacctgctcggtcaccttaggggtacctctaggtatttgtttttactttttgagacaaactcatataaagcttttaatttattccaccgtcacaagatttacagggttgaaaacaagatctctgggctgtcctaaaccaagcatggcggtcagtacccagcgttaaagtgtgctttgcaaagaagcacaatgttttgttccaaggatgcgaacatggcaccagccgtctgtctccgcccattctgcatcctcctgctctacttactttacacttacagtacaagcaggtatgtcaccttatttattgaaaatttgacctcatatcactctcaattacaggggctggcgaataataaaacaaactaaaggaaggatatggattataggaagccacgtgtaagatcaatggagtggagggctaaacaaggaaagtacacttcggatttcgaatcactcccagctaattctgttttctcggggaagcaacctgttctagatttggaatcactcccaaccaatcttcttattggggaaacaatttgtgccagatttggaatcactcccaactaattctgttttccaggggaaacaattaatccacgccacctaaacatgagtaaagtacgaagtacacgtactcaagtctatctcgctatatcggtgggtgttttgtgcgttagtaccatgtaaaaaataaatagatgacaaactgcattgctcgttaacttgcacgctagcactaagtacacctacagagatagatgtagagccctccatatgtggtgccttttacgcttaatgacaatgaaatgcgcgccagctgcattgctttttcacttgtgtgctgcaaatatgtacacgcgaacggatgtagagtattccatatataggtggtggtttgtacgtcaatacttgtctcatatccgccaaatgcatagtttttccactttgttgcggcaccaaatagacaaatcggactataaattgatacgcccaaggcaaagcccatagaacaagggactacacagacataaagagatccataacttaacct

SEQ ID NO: 4 - DNA, 1353 bp, Triticum aestivum
caacatacggcaactcatttgtaatttactgaacagttagtcagctaccatggtgtataagacctgctataccatattgttgtcgtatattcttatataagccttttctcataccatctcatgtatatgaaatctgtcatgcacgcaaacacgcaaccactgagggtacgccacgttgaactagcaggctcttgtttctcaatgcaagcaagctccgttggctgttaggattagatcctaaccaccttaggtgggagagaaggtgccgccgctgtggcatttgtcgtgtcttctagattacctgctcggtcaccttaggggtacctctaggtatttgtttttactttttgagacaaactcatataaagcttttaatttattccaccgtcacaagatttacagggttgaaaacaagatctccgggctgtcctaaaccaagcatggcggtcagtacccagcgttaaagtgtgctttgcaaagaagcacaatgttttgttccaaggatgcgaacatggcaccagccgtctgtctccgcccattctgcatcctcctgctctacttactttacacttacagtacaagcaggtatgtcaccttatttattgaaaatttgacctcatatcactctcaattacaggggctggcgaataataaaacaaactaaaggaaggatatggattatgggaagccatgtgtaagatcaatggagtggagggctaaacaaggaaagtacacttcggatttcgaatcactcccagctaatcctgttttctcggggaagcaacctgttctagatttggaatcactcccaaccaatcttcttattggggaaacaatttgtgccagatttggaatcactcccaactaattctgttttccaggggaaacaattaatccacgccacctaaacatgagtaaagtacgaagtacacgtactcaagtctatctcgctatatcggtgggtgttttgtgcgttagtaccatgtaaaaaataaatagatgacaaactgcattgcccgttaacttgcacgctagcactaagtacacctacagagatagatgtagagccctccatatgtggtgccttttacgcttaatgacaatgaaatgcgcgccagctgcattgctttttcacttgtgtgctgcaaatatgtacacgcgaacggatgtagagtattccatatataggtggtggtttgtacgtcaatacttgtctcatatccgccaaatgcatagcttttccactttgttgcgggcaccaaatagacacatcggactataaattgatacgcccaaggcaaagcccatagaacaagggactacacagacataaagagatccataacttaacct

SEQ ID NO: 5 - DNA, 1354 bp, Triticum aestivum
caacatacggcaactcatttgtaatttactgaacagttagtcagctaccatggtgtataagacctgctataccatattgttgtcgtatattcttatataagccttttctcataccatctcatgtatatgaaatctgtcatgcacgcaaacacgcaaccactgagggtacgccacgttgaactagcaggctcttgtttctcaatgcaagcaagctccgttggctgttaggattagatcctaaccatcttaggtgggagagaaggcgccgccgctgtggcatatgtcgtggtcttctagattacctgctcggtcaccttaggggtacctctaggtatttgtttttactttttgagacaaactcatataaagcttttaatttattccaccgtcacaagatttacagggttgaaaacaagatctccgggctgtcctaaaccaagcatggcggtcagtacccagcgttaaagtgtgctttgcaaagaagcacaatgttttgttccaaggatgcgaacatggcaccagccgtctgtctccgcccattctgcatcctcctgctctacttactttacacttacagtacaagcaggtatgtcgccttatttattgaaaatttgacctcatatcactctcaattacaggggctggcgaataataaaacaaactaaaggaaggatatggattatgggaagccatgtgtaagatcaatggagtggagggttaaacaaggaaagtacacttcggatttcgaatcactcccagctaattctgttttctcggggaagcaacctgttctagatttggaatcactcccaaccaatcttcttattggggaaacaatttgtgccggatttggaatcactcccaactaattctgttttccaggggaaacaattaatccacgccacctaaacatgagtaaagtacgaagtacacgtactcaagtctatctcgctatatcggtgggtgttttgtgcgttagtaccatgtaaaaaataaatagatgacaaactgcattgctcgttaacttgcacgctagcactaagtacacctacagagatagatgtagagccctccatatgtggtgccttttacgcttaatgacaatgaaatgcgcgccagctgcattgctttttcacttgtgtgctgcaaatatgtacacgcgaacggatgtagagtattccatatataggtggtggtttgtacgtcaatacttgtctcatatccgccaaatgcatagcttttccactttgttgcgggcaccaaatagacaaatcggactataaattgatacgcccaaggcaaagcccatagaacaagggactacacagacataaagagatccataacttaacct

SEQ ID NO: 6 - DNA, 1297 bp, Triticum aestivum
caacatacgacaactcatttgtaatttactgaacagttagtcagctaccatggtgtataagacctgctataccatgttgttgtcgtatattcttatataagccttttctcagaccatctcatgtatatgaaatctgtcatgcacgcaaacacgcaaccactgagggtacgccacgttgaactagcaggctcttgtttctcaatgcaagcaagctccgttggctgttaggattagatcctaaccaccttaggtgggagagaaggcgccgccgctgtggcatttgtcgtggtcttctagattacccgctcggtcaccttaggggtacctctaggtatttgtttttactttttgagacaaactcatataaagcctttaatttattccaccgtcacaagatttacagggttgaagacaagatctccgggctgtcctaaaccaagcatggcggtccgtacccagcgttaaagtgtgctttgcaaagaagcacaatgttttgttccaaggatgcgaacatggcaccagccgtctgtctccgcccattctgcatcctcctgctctacttactatacacttgcagtacaagcaggtatgtcaccttatctattgaaaatttgacctcatatcactctcaattacaggggctggcgaataataaaacaaactaaaggaaggatatggattatgggaagccatgtgtaagatcaatggagtggagggctgaacaaggaaagtacacttcggatttcgaatcactcccagctaattctgttttctcggggaagcaacctgttctagatttggaatcactcccaaccaatcttcttattggggaaacaatttgtgccagatttggaatcactcccaactaattctgttttccaggggaaacaattaatccacgccacctaaacatgagtaaagtacgaagtacacgtactcaagtctatctcgctatatcggtgggtgttttgtgcgttagtaccatgtaaaaaataaatagatgacaaactgcattgctcgttaacttgcacgctagcactaagtacacctacagagatagatgtagagccctccatatgtggtgccttttacgcttaatgacaatgaaatgcgcgccagctgcattgctttttcacttgtgtgctgcaaatatgtacacgcgaacggatgtagagtattccatatataggtggtggtttgtacgtcaatacttgtctcatatccgccaaatgcatagcttttccactttgttgcgggcaccaaatagacaaatcggactataaattgatacgcccaa

SEQ ID NO: 7 - DNA, 1354 bp, Triticum aestivum
caacatacggcaactcatttgtaatttactgaacagttagtcagctaccatggtgtataagacctgctataccatattgttgtcgtatattcttatataagccttttctcataccatctcatgtatatgaagtctgtcatgcacgcaaacacgcaaccactgagggtacgccacgttgaactagtaggctcttgtttctcaatgcaagcaagctccgttggctgttaggattagatcctaaccaccttaggtgggagagaaggcgccgccgctgtgacatttgtcgtggtcttctagattacctgctcggtcaccttaggggtacctctaggtatttgtttttactttttgagacaaactcatataaagcttttaatttatcccaccgtcacaagatttacagggttgaaaacaagatctccgggctgtcctaaaccaagcatggcggtcagtacccagcgttaaagtgtgctttgcaaagaagcacaatgttttgttccaaggatgcgaacatggcaccagccgtctgtctccgcccattctgcatcctcctgctctacttactttacacttacagtacaagcaggtatgtcaccttatttattgaaaatttgacctcatatcaccctcaattacaggggctggcgaataataaaacaaactaaaggaaggatatggattatgggaagccatgtgtaagatcagtggagtggagggctaaacaaggaaagtacacttcggatttcgaatcactcccagctaattctgttttctcggggaagcaacctgttctagatttggaatcactcccaaccaatcttcttattggggaaacaatttgtgccagatttggaatcactcccaactaattctgttttccaggggaaacaattaatccacgccacctaagcatgagtaaagtacgaagtacacgtactcaagtctatctcgctatatcggtgggtgttttgtgcgttagtaccatgtaaaaaataaatagacgacaaactgcattgctcgttaacttgcacgctagcactaagtacaccagcagagatagatgtagagccctccatatgtggtgccttttacgcttaatgacaatgaaatgcgcgccagctgcattgctttttcacttgtgtgctgcaaatatgtacacgcgaacggatgtagagtattccatatataggtggtggtttgtacgtcaatacttgtctcatatccgccaaatgcatagcttttccactttgttgcgggcaccaaatagacaaatcggactataaattgatacgcccaaggcaaagcccatagaacaagggactacacagacataaagagatccataacttaacct

SEQ ID NO: 8 - DNA, 1354 bp, Triticum aestivum
caacatacgacaactcatttgtaatttactgaacagttagtcagctaccatggtgtataagacctgctataccatattgttgtcgtatattcttatataagccttttctcataccatctcatgtatatgaaatctgtcatgcacgcaaacatgcaaccactgagggtacgccacgttgaactagcaggctcttgtttctcaatgcaagcaagctccgttggctgttaggattagatcctaaccaccttaggtgggagagaaggcgccgccgctgtggcatttgtcgtggtcttctagattacctgctcggtcaccttaggggtacctctaggtatttgtttttactttttgagacaaactcatataaagcttttaatttattccaccgtcacaagatttacagggttgaaaacaagatctccgggctgtcctaaaccaagcatggcggacagtacccagcgttaaagtgtgctttgcaaagaagcacaatgttttgttccaaggatgcgaacatggcaccagccgtctgtctccgcccattccgcatcctcctgctctacttactttacacttacagtacaagcaggtatgtcaccttatttattgaaaatttgacctcatatcactctcaattacaggggctggcgaataataaaacaaactaaaggaaggatatggattatgggaagccatgtgtaagatcaatggagtggagggctaaacaaggaaagtacacttcggatttcgaatcactcccagctaattctgttttctcggggaagcaacctgttctagatttggaatcactcccaaccaatcttcctattggggaaacaatttgtgccagatttggaatcactcccaactaattctgttttccaggggaaacaattaatccacgccacctaaacatgagtaaagtacgaagtacacgtactcaagtctatctcgctatatcggtgggtgttttgtgcgttagtaccatgtaaaaaataaatagatgacaaactgcattgctcgttaacttgcacgctagcactaagtacacctacagagatagatgtagagccctccatatgtggtgccttttacgcttaatgacaatgaaatgcgcgccagctgcattgctttttcacttgtgtgctgcaaatatgtacacgcgaacggatgtagagtattccatatataggtggtggtttgtacgtcaatatttgtctcatatccgccaaatgcatagcttttccactttgttgcgggcaccaaatagacaaatcggactataaattgatacgcccaaggcaaagcccatagaacaagggactacacagacataaagagatccataacttaacct

SEQ ID NO: 9 - DNA, 1353 bp, Triticum aestivum
caacatacggcaactcatttgtaatttactgaacagttagtcagctaccatggtgtataagacctgctataccatattgttgtcgtatattcttatataagccttttctcataccatctcatgtatatgaaatctgtcatgcacgcaaacacgcaaccaccgagggtacgccacgttgaactagcaggctcttgtttctcaatgcaagcaagctccgttggctgttaggattagatcctaaccaccttaggtgggagagaaggcgccaccgctgtggcatttgtcgtggtcttctagattacctgctcggtcaccttaggggtacctctaggtatttgtttttactttttgagacaaactcatataaagcttttaatttattccaccgtcacaagatttacggggttgaaaacaagatctccgggctgtcctaaaccaagcatggcggtcagtacccagcgttaaagtgtgctttgcaaagaagcacaatgttttgttccaaggatgcgaacatagcaccagccgtctgtctccgcccattctgcatcctcctgctctacttactttacacttacagtacaagcaggtatgtcaccttatttattgaaaatttgacctcatatcactctcaattacaggggctggcgaataataaaacaaactaaaggaaggatatggattatgggaagccatgtgcaagatcaatggagtggagggctaaacaaggaaagtacacttcggatttcgaatcactcccagctaattctgttttctcagggaagcaacctgttctagatttggaatcactcccaaccaatcttcttattggggaaacaatttgtgccagatttggaatcactcccaactaattctgttttccaggggaaacaattaatccacgccacctaaacatgagtaaagtacgaagtacacgtgctcaagtctatctcgctatatcggtgggtgttttgtgcgttagtaccatgtaaaaaataaatagatgacaaactgcattgctcgttaacttgcacgctagcactaagtacaccaacagagatagatgtagagccctccatatgtggtgccttttacgcttaatgacaatgaaatgcgcgccagctgcattgctttttcacttgtgtgctgcaaatatgtgcacgcgaacggatgtagagtattccatatataggtggtggtttgtacgtcaatacttgtctcatatccgccaaatgcatagctttccactttgttgcgggcaccaaatagacaaatcggactataaattgatacgcccaaggcaaagcccatagaacaagggactacacagacataaagagatccataacttaacct

SEQ ID NO: 10 - DNA, 1354 bp, Triticum aestivum
caacatacggcaactcatttgtaatttactgaacagttagtcagctaccgtggtgtataagacctgctataccatattgttgtcgtatattcttatataagccttttctcataccatctcatgtatatgaaatctgtcatgcacgcaaacacgcaaccactgagggtacgccacgttgaactagcaggctcttgtttctcaatgcaagcaagctccgttggctgttaggattagatcctaaccaccttaggtgggagagaaggcgccgccgctgtggcattcgtcgtagtcttctagattacctgctcggtcaccttaggggtacctctaggtatttgtttttactttttgagacgaactcatataaagcttttaatttattccaccgtcacaagatttacagggttgaaaacaagatctccgggctgtcctaaaccaagcatggcggccagtacccagcgttaaagtgtgctttgcaaagaagcacgatgttttgttccaaggatgcgaacatggcaccagccgtctgtctccgcccattctgcatcctcctgctctacttactttacacttacagtacaagcaggtatgtcaccttatttattgaaaatttgacctcatatcactctcaattacaggggctggcgaataataaaacaaactaaaggaaggatatggattatgggaagccatgtgtaagatcagtggagtggagggctaaacaaggaaagtacacttcggatttcgaatcactcccagctaattctgttttctcggggaagcaacctgttctagatttggaatcactcccaaccaatcttcttattggggaaacaatttgtgccagatttggaatcactcccaactaattctgttttccaggggaaacaattaatccacgccacctaaacatgagtaaagtacgaagtacacgtactcaagtctatctcgctatatcggtgggtgttttgtgcgctagtaccgtgtaaaaaataaatagatgacaaactgcattgctcgctaacttgcacgcttgcactaagtacaccaacagaggtagatgtagagccctccatatgtggtgccttttacgcttaatgacaatgaaatgcgcgccagctgcattgctttttcacttgcgtgctgcaaatatgtacacgcgaacggatgtagagtgttccatatataggtggtggtttgtacgtcaatacttgtctcatatccgccaaatgcatagcttttccactttgttgtgggcaccaaatagacaaatcggactataaattgatacgcccaaggcaaagcccatagaacaagggactacacagacataaagagatccataacttaacct

SEQ ID NO: 11 - DNA, 1354 bp, Triticum aestivum
caacatacggcaactcatttgtaatttactgaacagttagtcagctaccatggtgtataagacctgctataccatattgttgtcgtatattcttatataagccttttctcataccatctcatgtatatgaaatctgtcatgcacgcaaacacgcaaccactgagggtacgccacgttgaactagcaggctcttgtttctcaatgcaagcaagctccgttggctgttaggattagatcctaaccaccttaggtgggagagaaggcgccgccgctgtggcatttgtcgtggtcttctagattacctgctcggtcaccttaggggtacctctaggtatttgtttttactttttgagacaaactcatataaagcctttaatttactccaccgtcacaagatttacagggttgaaaacaagatctccgggctgtcctaaaccgagcatggcggtcagtacccagcgttaaagtgtgctttgcaaagaagcacagtgttttgttccaaggatgcgaacatggcaccagccgtctgtctccgcccattctgcatcctcctgctctacttactttacacttacagtacaagcaggtatgtcaccttatttatcgaaaatttgacctcatatcactctcaattacaggggctggcgaataataaaacaaactaaaggaaggatatggattatgggaagccatgtgtaagatcagtggagtggagggctaaacaaggaaagtacacttcggatttcgaatcactcccagctaattctgttttctcggggaagcaacctgttctagatttggaatcactcccaaccaatcttcttattggggaaacaatttgtgccagatttggaatcactcccaactaattctgttttccaggggaaacaattaatccacgccacctaaacatgagtaaagtacgaagtacacgtactcaagtctatctcgctatatcggtgggtgttttgtgcgttagtaccatgtaaaaaataaatagatgacaaactgcattgctcgttaacttgcacgctagcactaagtacaccaacagagatagatgtagagccctccatatgtggtgccttttacgcttaatgacaatgaaatgcgagccagctgcattgctttttcgcttgtgtgctgcaaatatgtacacgcgaacggatgtagagtattccatatataggtggtggtttgtacgtcaatacttgtctcatatccgccaaatgcatagcttttccactttgttgcgggcaccgaatagacaaatcggactataaattgatacgcccaaggcaaagcccatagaacaagggactacacagacataaagagatccataacttaacct

SEQ ID NO: 12 - DNA, 1354 bp, Triticum aestivum
caacatacggcaactcatttgtaatttactgaacagttagtcagctaccatggtgtataagacctgctataccatattgttgtcgtatattcttatataagccttttctcataccatctcatgtatatgaaatctgtcatgcacgcaaacacgcaaccactgagggtacgccacgttgaactagcaggctcttgtttctcaatgcaagcaagctccgttggctgttaggattagatcctaaccaccttaggtgggagagaaggcgccgccgccgtggcatttgtcgtggtcttctagattacctgctcggtcaccttaggggtacctctaggtatttgtttttactttttgagacaaactcatataaagcttttaatttattccaccgtcacaagatttacagggttgaaaacaagatctccgggctgtcctaaaccaagcatggcggtcagtacccagcgttaaagtgtgctttgcaaagaagcacaatgttttgttccaaggatgcgaacatggcaccagccgtctgtctccgcccattctgcatcctcctgctctacttactttacacttacagtacaagcaggtatgtcaccttatttattgaaaatttgacctcatatcactctcaattacaggggctggcgaataataaaacaaactaaaggaaggatatggattatgggaagccatgtgcaagatcagtggagtggagggctaaaccaggaaagtacacttcggatttcgaatcactcccagccgattctgttttctcggggaagcaacctgttctagatttggaatcactcccaaccaatcttcttattggggaaacaatttgtgccagatttggaatcactcccaactaattctgttttccaggggaaacaattaatccacgccacctaaacatgagtaaagtacgaagtacacgtactcaagtctatctcgctatatcggtgggtgttttgtgcgttagtaccatgtaaaaaataaatagatgacaaactgcattgctcgttaacttgcacgctagcactaagtacaccaacagagatagatgtagagccctccatatgtggtgccttttacgcttaatgacaatgaaatgcgcgccagctgcattgctttttcacttgtgtgctgcaaatatgtacacgcgaacggatgtagagtattccatatataggtggtggtttgtacgtcaatacttgtctcatatccgccaaatgcatagcttttccactttgttgcgggcaccaaatagacgaatcggactataaattgatacgcccaaggcaaagcccatagaacaagggactacacagacataaagagatccataacttaacct

SEQ ID NO: 13 - DNA, 1354 bp, Triticum aestivum
caacatacggcaactcatttgtaatttactgaacggttagtcagctaccatggtgtataagacctgctataccatattgttgtcgtatattcttatataagccttttctcataccatctcatgtatatgaaatctgtcatgcacgcaagcacgcaaccactgagggtacgccacgttgaactagcaggctcttgtttctcaatgcaagcaagctccgttggctgttaggattagatcctaaccaccttaggcgggagagaaggcgccgccgctgtggcatttgtcgtggtcttctagattacctgctcggtcaccttaggggtacctctaggtatttgtttttactttttgagacaaactcatataaagcttttaatttactccaccgtcacaagatttacagggttgaaaacaagatctccgggctgtcctaaaccaagcatggcggtcagtacccagcgttaaagtgtgctttgcaaagaagcacaatgttttgttccaaggatgcgaacatggcaccagccgtctgtctccgcccattctgcatcctcctgctctacttactttacacttacagtacgagcaggtatgtcaccttatttattgaaaatttgacctcatatcactctcaattacaggggctggcgaataataaaacaaactaaaggaaggatatggattatgggaagccatgtgtaagatcaatggagtggagggctaaacaaggaaagtacacttcggatttcgaatcactcccagctaattctgttttctcagggaagcaacctgttctagatttggaatcactcccaaccaatcttcttattggggaaacaatttgtgccagatttggaaccactcccaactaattctgttttccaggggaaacaattaatccacgccacctaaacatgagtaaagtacgaagtacacgtactcaagtctatctcgctatatcggtgggtgttttgtgcgttagtaccatgtaaaaaataaatagatgacaaactgcattactcgttaacttgcacgctagcactaagtacacctacagagatagatgtagagcccgccatatgtggtgccttttacgcttaatgacaacgaaatgcgcgccagctgcattgctttttcacttgtgtgctgcaaatatgtacacgcgaacggatgtagagtattccatatataggtggtggtttgtacgtcaatacttgtctcatatccgccaaatgcatagtttttccactttgttgcgggcaccaaatagacaaatcggactataaattgatatgcccaaggcaaagcccatagaacaagggactacacagacataaagagatccataacttaacct

SEQ ID NO: 14 - DNA, 1353 bp, Triticum aestivum
caacttacggcaactcatttgtaatttactgaacagttagtcagctaccatggtgtataagacctgccataccatattgttgtcgtatattcttatataagccttttctcataccatctcatgtatatgaaatctgtcatgcacgcaaacacgcaaccactgagggtacgccacgttgaactagcaggctcttgtttctcaatgcaagcaagctccgttggctgttaggattagatcctaaccaccttaggtgggagagaaggcgccgccgctgtggcatttgtcgtggtcttctagattacctgctcggtcaccttaggggtacctctaggtatttgtttttactttttgagacaaactcatataaagcttttaatttattccaccgtcacaagatttacagggttgaaaacaagatctccgggctgtcttaaaccaagcatggcggtcagtacccagcgttaaagtgtgctttgcaaagaagcacaatgttttgttccaaggatgcgaacatggtaccagccgtctgtctccgcccattctgcatcctcctgctctacttactttacacttacagtacaagcaggtatgtcaccttatttattgaaaatttgacctcatatcactctcaattacaggggctggcgaataataaaacaaactaaaggaaggatatggattatgggaagccatgtgtaagattaatggagtggagggctaaacaaggaaagtacacttcggatttcgaatcactcccagctaattctgttttctcggggaagcaacctgttctagatttggaatcactcccaaccaatcttcttattggggaaacaatttgtgccagatttggaatcactcccaactaattctgttttccaggggaaacaattaatccacgccacctaaacatgagtaaagtacgaagtacacgtactcaagtctatctcgctatatcggtgggtgttttgtgcgttagtgccatgtaaaaaataaatagatgacaaactgcattgctcgttaacttgcacgctagcactaagtacacctacagagatagatgtagagccctccatatgtggtgccttttacgcttaatgacaatgaaatgcgcgccagctgcattgctttttcgctcgtgtgctgcaaatatgtgcacgcgaacggatgtagagtattccatatataggtggtggtttgtacgtcaatacttgtctcatatccgccaaatgcatagcttttccactttgttgcgggcaccaaatagacaaatcggactataaattgatacgcccaggcaaagcccatagaacaagggactacacagacataaagagatccataacttaacct

SEQ ID NO: 15 - DNA, 1354 bp, Triticum aestivum
caacatacgacaactcatttgtaatttactgaacagttagtcagctaccatggtgtataagacctgctataccatatcgttgtcgtatattcttatataggccttttctcataccatctcatgtatatgaaatctgtcatgcacgcaaacacgcaaccactgagggtacgccacgttgaactagcaggctcttgtttctcaatgcaagcaagctccgttggctgttaggattagatcctaaccaccttaggtgggagagaaggcgccgctgctgtggcatttgtcgtggtcttctagattacctgctcggtcaccttaggggtacctctaggtatttgtttttactttttgagacaaactcatataaagcttttaatttattccaccgtcacaagatttacagggttgaaaacaagatctccgggctgtcctaaaccaagcatggcggtcagtacccagcgttaaagtgtgctttgcaaagaagcacaatgttttgttccaaggatgcgaacatggcaccagccgtctgtctccgcccattctgcatcctcctgctctacttactttacacttacagtacaagcaggtatgtcaccttatttattgaaaatttgacctcatatcactctcaattacaggggctggcgaataataaaacaaactaaaggaaggatatggattatgggaagccatgtgtaagatcaatggagtggagggctaaacaaggaaagtacacttcggatttcgaatcactcccagctaattctgttttctcggggaagcaacctgttctagatttggaaccactcccaaccaatcttcttattggggaaacaatttgtgccagatttggaatcactcccaactaattctgttttccaggggaaacagttaatccacgccacctaaacatgagtaaagtacgaagtacatgtactcaagtctatctcgcaatatcggcgggtgttttgtgcgttagtaccatgtaaaaaataaatagatgacaaactgcattgctcgttaacttgcacgctagcactaagtacacctacagagatagatgtagagccctccatatgtggtgccttttacgcttaatgacaatgaaatgcgcgccagctgcattgctttttcacttgtgtgctgcaaatatgtacacgcgaacggatgtagagtattccatatataggtggtggtttgtacgtcaatacttgtctcatatccgccaaatgcatagcttttccactttgttgcgggcaccaaatagacaaatcggactataaattgatacgcccaaggcaaagcccttagaacaagggactacacagacataaagagatccataacttaacct

SEQ ID NO: 16 - DNA, 505 bp, Triticum aestivum
gagaacaagggactacacagacataaagagatccataacttaacctgaagagtgtgacgagataggcagccatgttcttctccacaaagatgtgtgttgctaccatcatggtgctagccctgacgctctcgcctcatggcaccgttgacgccggccacctctcttcaaactggggctcttgtccagatggacagtcagtgcaatgcattgggagaccgccattctgcaagtgtgtaccaaaccttcagtttgtggatcgccagcgtactgtgtacaacatgggagctgcccgtgcatagtaactagagtttctagtaatatactatagcaataagagtcatgcagcatccctgcatgcatcgccagaaaacttctgtacaacggaaagcgtgtcaactgaacttgatatatgtatgcaccattgcatgcatgttgttccgtgtagtaccagaaaataaaataagggctgctactatgtatgcaggccatttgcttttagcaasaa

SEQ ID NO: 17 - DNA, 522 bp, Triticum aestivum
gagaacaagggactacacagacataaagagatccataacttaacctgaagagtgtgacgagataggcagccatgttcttctccacaaagatgtgtgttgctaccatcatggtgctagccctgacgctctcgcctcatggcaccgttgacgccggccacctctcttcaaactggggctcttgtccagatggacagtcagtgcaatgcattgggagaccgccattctgcaagtgtgtaccaaaccttcagtttgtggatcgccagcgtactgtgtacaacatgggagctgcccgtgcatagtaactagagtttctagtaatatactatagcaataagagtcatgcagcatccctgcatgcatcgccagaaaacttctgtacaacggaaagcgtgtcaactgaacttgatatatgtatgcaccattgcatgcatgttgttccgtgtagtaccagaaaataaaataagggctgctactatgtatgcaggccatttgcttttagcyaywgaawmaaaawatrarwra

SEQ ID NO: 18 - DNA, 526 bp, Triticum aestivum
gagaacaagggactacacagacataaagagatccataacttaacctgaagagtgtgacgagatgggcagccatgttcttctccacaaagatgtgtgttgctaccatcatggtgctagccctgacgctctcgcctcatggcaccgttgacgccggccacctctcttcaaactggggctcttgtccagatggacagtcagtgcaatgcattgggagaccgccattctgcaagtgtgtaccaaaccttcagtttgtggatcgccagcgtactgtgtacaacatgggagctgcccgtgcatagtaactagagtttctagtaatatactatagcaataagagtcatgcagcatccctgcatgcatcgccagaaaacttctgtacaacggaaagcgtgtcaactgaacttgatatatgtatgcaccattgcatgcatgttgttccgtgtagtaccagaaaataaaataagggctgctactatgtatgcaggccatttgcttttaaaaaaaaaaaaaaaaaaaaaaaaaaaaa

SEQ ID NO: 19 - DNA, 527 bp, Triticum aestivum
ggaacaagggactacacagacataaagagatccataacttaacctgaagagtgtgacgagataggcagccatgttcttctccacaaagatgtgtgttgctaccatcatggtgctagccctgacgctctcgcctcatggcaccgttgacgccggccacctctcttcaaactggggctcttgtccagatggacagtcagtgcaatgcattgggagaccgccattctgcaagtgtgtaccaaaccttcagtttgtggatcgccagcgtactgtgtacaacatgggagctgcccgtgcatagtaactagagtttctagtaatatactatagcaataagagtcatgcagcatccctgcatgcatcgccagaaaacttctgtacaacggaaagcgtgtcaactgaacttgatatatgtatgcaccattgcatgcatgttgttccgtgtagtaccagaaaataaaataagggctactactatgtatgcaggccatttgcttttgaaaaaaaaaaaaaaaaaaaaaaaaaaaaaa

SEQ ID NO: 20 - DNA, 526 bp, Triticum aestivum
ggaacaagggactacacagacataaagagatccataacttaacctgaagagtgtgacgagataggcagccatgttcttctccacaaagatgtgtgttgctaccatcatggtgctagccctgacgctctcgcctcatggcaccgttgacgccggccacctctcttcaaactggggctcttgtccagatggacagtcagtgcaatgcattgggagaccgccattctgcaagtgtgtaccaaaccttcagtttgtggatcgccagcgtactgtgtacaacatgggagctgcccgtgcatagtaactagagtttctagtaatatactatggcaataagagtcatgcagcatccctgcatgcatcgccagaaaacttctgtacaacggaaagcgtgtcaactgaacttgatatatgtatgcaccattgcatgcatgttgttccgtgtagtaccagaaaataaaataagggctgctactatgtatgcaggccatttgcttttaagaaaaaaaaaaaaaaaaaaaaaaaaaaa

SEQ ID NO: 21 - DNA, 524 bp, Triticum aestivum
ggaacaagggactacacagacataaagagatccataacttaacctgaagagtgtgacgagataggcagccatgttcttctccacaaagatgtgtgttgctaccatcatggtgctagccctgacgctctcgcctcatggcaccgttgacgccggccacctctcttcaaactggggctcttgtccagatggacagtcagtgcaatgcattgggagaccgccattctgcaagtgtgtaccaaaccttcagtttgtggatcgccagtgtactgtgtacaacatgggagctgcccgtgcatagtaactagagtttctagtaatatactatagcaataagagtcatgcagcatccctgcatgcatcgccagaaaacttctgtacaacggaaagcgtgtcaactgaacttgatatatgtatgcaccattgcatgcatgttgttccgtgtagtaccagaaaataaaataagggctgctactatgtatgcaggccatttgcttttagcaaagaaaaaaaacaaaaaaaaaaaa

SEQ ID NO: 22 - DNA, 522 bp, Triticum aestivum
ggaacaagggactacacagacataaagagatccataacttaacctgaagagtgtgacgagataggcagccatgttcttctccacaaagatgtgtgttgctaccatcatggtgctagccctgacgctctcgcctcatggcaccgttgacgccggccacctctcttcaaactggggctcttgtccagatggacagtcagtgcaatgcattgggagaccgccattctgcaagtgtgtaccaaaccttcagtttgtggatcgccagcgtactgtgtacaacatgggaactgcccgtgcatagtaactagagtttctagtaatatactatagcaataagagtcatgcagcatccctgcatgcatcgccagaaaacttctgtacaacggaaagcgtgtcaactgaacttgatatatgtatgcaccattgcatgcatgttgttccgtgtagtaccagaaaataaaataagggctgctactatgtatgcaggccatttgcttttaacaaaaaaaaaaaaaaaaaaaaaaa

SEQ ID NO: 23 - DNA, 506 bp, Triticum aestivum
ggaacaagggactacacagacataaagagatccataacttaacctgaagagtgtgacgagataggcagccatgttcttctccacaaagatgtgtgttgctaccatcatggtgctagccctgacgctctcgcctcatggcaccgttgacgccggccacctctcttcaaactggggctcttgtccagatggacagtcagtgcaatgcattgggagaccgccattctgcaagtgtgtaccaaaccttcagtttgtggatcgccagcgtactgtgtacaacatgggagctgcccgtgcatagtaactagagtttctagtaatatactatagcaataagagtcatgcagcatccctgcatgcatcgccagaaaacttctgtacaacggaaagcgtgtcaactgaacttgatatatgtatgcaccattgcatgcatgttgttccgtgtagtaccagaaaataaaataagggctgctactatgtatgcaggccatttgcttttaaaaaraaaa

SEQ ID NO: 24 - DNA, 481 bp, Triticum aestivum
ggaacaagggactacacagacataaagagatccataacttawgctgaagagtgtgacgagataggcagccatgttcttctccacaaagatgtgtgttgctaccatcatggtgctagccctgacgctctcgcctcatggcaccgttgacgccggccacctctcttcaaactggggctcttgtccagatggacagtcagtgcaatgcattgggagaccgccattctgcaagtgtgtaccaaaccttcagtttgtggatcgccagcgtactgtgtacaacatgggagctgcccgtgcatagtaactagagtttctagtaatatactatagcaataagagtcatgcagcatccctgcatgcatcgccagaaaacttctgtacaacggaaagcgtgtcaactgaacttgatatatgtatgcaccattgcatgcatgttgttccgtgtagtaccagaaaataaaataagggctgctactatgtatgc

SEQ ID NO: 25 - DNA, 520 bp, Triticum aestivum
gggactacacagacataaagagatccataacttaacctgaagagtgtgacgagataggcagccatgttcttctccacaaagatgtgtgttgctaccatcatggtgctagccctgacgctctcgcctcatggcaccgttgacgccggccacctctcttcaaactggggctcttgtccagatggacagtcagtgcaatgcattgggagaccgccattctgcaagtgtgtaccaaaccttcagtttgtggatcgccagcgtactgtgtacaacatgggagctgcccgtgcatagtaactagagtttctagtaatatactatagcaataagagtcatgcagcatccctgcatgcatcgccagaaaacttctgtacaacggaaagcgtgtcaactgaacttgatatatgtatgcaccattgcatgcatgttgttccgtgtagtaccagaaaataaaataagggctgctactatgtatgcaggccatttgcttttaggaaaaaaaaaaaaaaaaaaaaaaaaaaaa

SEQ ID NO: 26 - DNA, 490 bp, Triticum aestivum
gacaagggactacacagacataaagagatccataacttaacctgaagagtgtgacgagataggcagccatgttcttctccacaaagatgtgtgttgctaccatcatggtgctagccctgacgctctcgcctcatggcaccgttgacgccggccacctctcttcaaactggggctcttgtccagatggacagtcagtgcaatgcattgggagaccgccattctgcaagtgtgtaccaaaccttcagtttgtggatcgccagcgtactgtgtacaacatgggagctgcccgtgcatagtaactagagtttctagtaatatactatagcaataagagtcatgcagcatccctgcatgcatcgccagaaaacttctgtacaacggaaagcgtgtcaactgaacttgatatatgtatgcaccattgcatgcatgttgttccgtgtagtaccagaaaataaaataagggctgctactatgtatgcaggccaataaa

SEQ ID NO: 27 - DNA, 431 bp, Triticum aestivum
agagtgtgacgagataggcagccatgttcttctccacaaagatgtgtgttgctaccatcatggtgctagccctgacgctctcgcctcatggcaccgttgacgccggccacctctcttcaaactggggctcttgtccagatggacagtcagtgcaatgcattgggagaccgccattctgcaagtgtgtaccaaaccttcagtttgtggatcgccagcgtactgtgtacaacatgggagctgcccgtgcatagtaactagagtttctagtaatatactatagcaataagagtcatgcagcatccctgcatgcatcgccagaaaacttctgtacaacggaaagcgtgtcaactgaacttgatatatgtatgcaccattgcatgcatgttgttccgtgtagtaccacaaaaaaaaaaaaaaaaaaaaaaaaaaaa

SEQ ID NO: 28 - protein, 75 aa, Triticum aestivum
MFFSTKMCVATIMVLALTLSPHGTVDAGHLSSNWGSCPDGQSVQCIGRLPFCKCVPNLQFVDRQRTVYNMGAARA

SEQ ID NO: 29 - DNA, 1255 bp, Triticum aestivum
agccttttctcataccatctcatgtatatgaaatctgtcatgcacgcaaacacgcaaccactgagggtacgccacgttgaacttgtttctcaatgcaagcaagctccgttggctgttaggattagatcctactagcaggctgtgggagagaaggcgccgccgctgtggcatttgtcgtggtcttctagattaccaccttagacctgctcggtcaccttaggggtacctctaggtatttgtttttactttttgagacaaactcatataaagcttttaatttattccaccgtcacaagatttacagggttgaaaacaagatctccgggctgtcctaaaccaagcatggcggtcagtacccagcgttaaagtgtgctttgcaaagaagcacaatgttttgttccaaggatgcgaacatggcaccagccgtctgtctccgcccattctgcatcctcctgctctacttactttacacttacagtacaagcaggtatgtcaccttatttattgaaaatttgacctcatatcactctcaattacaggggctggcgaataataaaacaaactaaaggaaggatatggattatgggaagccatgtgtaagatcaatggagtggagggctaaacaaggaaagtacacttcggatttcgaatcactcccagctaattctgttttctcggggaagcaacctgttctagatttggaatcactcccaaccaatcttcttattggggtgccagatttggaatcactcccaactaattctgttttccaggggaaacaattaatccacgaaacaatttgccacctaaacatgagtaaagtacgaagtacacgtactcaagtctatctcgctatatcggtgggtgttttgtgcgttagtaccatgtaaaaaataaatagatgacaaactgcattgctcgttaacttgcacgctagcactaagtacacctacagagatagatgtagagccctccatatgtggtgccttttacgcttaatgacaatgaaatgcgcgccagctgcattgctttttcacttgtgtgctgcaaatatgtacacgcgaacggatgtagagtattccatatataggtggtggtttgtacgtcaatacttgtctcatatccgccaaatgcatagcttttccactttgttgcgggcaccaaatagacaaatcggactataaattgatacgcccaaggcaaagcccatagaacaagggactacacagacataaagagatccataacttaacct

SEQ ID NO: 30 - DNA, 21 bp, Triticum aestivum
catgttgttccgtgtagtacc

SEQ ID NO: 31 - DNA, 26 bp, Triticum aestivum
ggctagcaccatgatggtagcaacac

SEQ ID NO: 32 - DNA, 26 bp, Triticum aestivum
tggctgcctatctcgtcacactcttc

SEQ ID NO: 33 - DNA, 10 bp, Triticum aestivum
  misc_feature (4)..(7): n is a, c, g, or t
caannnnatc

SEQ ID NO: 34 - DNA, 19 bp, Triticum aestivum
  misc_feature (3)..(14): n is a, c, g, or t
ccnnnnnnnnnnnnccacg

SEQ ID NO: 35 - DNA, 11 bp, Triticum aestivum
  misc_feature (7)..(9): n is a, c, g, or t
rgtgacnnngc

SEQ ID NO: 36 - DNA, 10 bp, Triticum aestivum
cwwwwwwwwg

SEQ ID NO: 37 - DNA, 10 bp, Triticum aestivum
cwwwwwwwwg

SEQ ID NO: 38 - DNA, 10 bp, Triticum aestivum
  misc_feature (4)..(7): n is a, c, g, or t
caannnnatc

SEQ ID NO: 39 - DNA, 10 bp, Triticum aestivum
ttwtwttwtt

SEQ ID NO: 40 - DNA, 19 bp, Triticum aestivum
  misc_feature (3)..(14): n is a, c, g, or t
ccnnnnnnnnnnnnccacg

SEQ ID NO: 41 - DNA, 467 bp, Triticum aestivum
aagagatccataacttaacctgaagagtgtgacgagataggcagccatgttcttctccacaaagatgtgtgttgctaccatcatggtgctagccctgacgctctcgcctcatggcaccgttgacgccggccacctctcttcaaactggggctcttgtccagatggacagtcagtgcaatgcattgggagaccgccattctgcaagtgtgtaccaaaccttcagtttgtggatcgccagcgtactgtgtacaacatgggagctgcccgtgcatagtaactagagtttctagtaatatactatagcaataagagtcatgcagcatccctgcatgcatcgccagaaaacttctgtacaacggaaagcgtgtcaactgaacttgatatatgtatgcaccattgcatgcatgttgttccgtgtagtaccagaaaataaaataagggctgctactatgtatgcaggccatttg

SEQ ID NO: 42 - DNA, 467 bp, Triticum aestivum; identical to SEQ ID NO: 41

SEQ ID NO: 43 - DNA, 362 bp, Triticum aestivum
ataaagagatccataacttaacctgaagagtgtgacgagataggcagccatgttcttctccacaaagatgtgtgttgctaccatcatggtgctagccctgacgctctcgcctcatggcaccgttgacgccggccacctctcttcaaactggggctcttgtccagatggacagtcagtgcaatgcattgggagaccgccattctgcaagtgtgtaccaaaccttcagtttgtggatcgccagcgtactgtgtacaacatgggagctgcccgtgcatagtaactagagtttctagtaatatactatagcaataagagtcatgcagcatccctgcatgcatcgccgaaaaaaaaaaaaaaaaaaa

SEQ ID NO: 44 - DNA, 362 bp, Triticum aestivum; identical to SEQ ID NO: 43

(19) United States
(12) Patent Application Publication
(10) Pub. No.: US 2012/0240286 A1
(43) Pub. Date: Sep. 20, 2012

(54) NUCLEIC ACID PROMOTER SEQUENCES THAT CONTROL GENE EXPRESSION IN PLANTS

(75) Inventors: Robert J. Henry, Tuckombil (AU); Allison C. Crawford, Coogee (AU)

(73) Assignee: Grains Foods CRC Ltd., North Ryde New South Wales (AU)

(21) Appl. No.: 12/444,302

(22) PCT Filed: Oct. 4, 2007
(86) PCT No.: PCT/AU07/01479

§ 371 Date: Mar. 24, 2010

(30) Foreign Application Priority Data
Oct. 4, 2006 (AU) 2006905476

(51) Int. Cl. A01H 5/00; C12N 5/10; A01H 1/06; C12N 15/11; C07K 14/415; C07K 16/16; C12N 15/82; C12N 15/113

(52) U.S. Cl. 800/278; 435/320.1; 435/419; 435/468; 536/24.1; 536/23.1; 530/370; 530/387.9; 800/298; 800/320.3

(57) ABSTRACT

An isolated nucleic acid sequence comprising a nucleotide sequence which corresponds to a promoter-active region of a DNA sequence is provided herein, wherein the isolated nucleic acid sequence is derived from a cereal seed. The isolated nucleic acid sequences are useful for the control of high levels of gene expression within specific tissues of a cereal seed. Also provided are genetic constructs comprising the isolated nucleic acid sequences (and variants thereof) operably-linked to a transcribable sequence, methods of producing a recombinant protein using said genetic constructs and methods of facilitating target expression to a plant endosperm.

### FIELD OF THE INVENTION

This invention relates to gene expression. More particularly, this invention relates to nucleic acid promoter sequences which control expression of proteins in plants.

### BACKGROUND TO THE INVENTION

The cereal grain endosperm is the single major source of carbohydrates in the human diet. Genetic modification of the endosperm therefore provides potential opportunities for further increasing the nutritional and/or economic value of cereal crops and associated products (Lamacchia et al. 2001; Shewry and Jones 2005). One particular avenue of research focuses on the manipulation of cereal seeds to produce pharmaceutical proteins such as replacement human proteins and antibodies. Downstream extraction of pharmaceutical proteins from seeds is also relatively straightforward compared to other plant tissues, with fewer extraneous compounds present that may potentially interfere in this process

Wheat is an attractive crop for developing a transgene platform due to its lower production costs compared to maize and rice. While some recombinant proteins have been produced at high levels in the wheat endosperm (reviewed by Stoger et al. 2005) successful production of antibodies and other pharmaceutical or industrial proteins has not yet been achieved. This has been attributed to the limited availability of promoter sequences that have the appropriate level and specificity of gene expression. In this regard, promoters often function in a similar manner across species boundaries, however this does not appear to be true for the cereal endosperm which has undergone significant divergence across species (Drea et al. 2005).

### SUMMARY OF THE INVENTION

Despite the enormous potential of wheat seed as a recombinant protein expression system, currently only a very limited number of promoters have been characterized for this purpose.

The present invention is broadly directed to isolated nucleic acid sequences from a cereal seed which are useful for control of high levels of gene expression within specific tissues of a cereal seed.

In a first aspect, the invention provides an isolated nucleic acid comprising a nucleotide sequence which corresponds to a promoter-active region of a gene comprising a transcribable DNA sequence encoding SEQ ID NO:28.

In one embodiment, the promoter-active region comprises a nucleotide sequence as set forth in SEQ ID NO: 1, or a variant thereof.

Preferably, the variant has at least 50%, 55%, 60%, 65%, 70%, 75%, 80%, 85% or 90% and more preferably at least 95%, 96%, 97%, 98% or 99% sequence identity to the nucleotide sequence as set forth in SEQ ID NO: 1.

Preferably, the transcribable DNA sequence is obtained from a seed. More preferably, the seed is derived from a cereal such as, but not limited to, wheat.

Even more preferably, the cereal is wheat.

Transcription of a transcribable DNA sequence may be either constitutive or, alternatively, tissue-specific. Advantageously, the transcribable DNA sequence is selected from the group consisting of the nucleotide sequences set forth in SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26 and SEQ ID NO: 27.

Preferably, the transcribable DNA sequence is highly transcribed in an endosperm of a cereal.

More preferably, the transcribable DNA sequence is highly transcribed in an endosperm of wheat

The invention also readily contemplates a nucleotide sequence which corresponds to a promoter-active fragment of the promoter-active region.

A promoter-active fragment may, for the purpose of regulating transcription, include a number of control elements such as, but not limited to, a TATA box, an INR element and transcription factor binding sites.

Preferably, the promoter-active fragment comprises at least one element from the group set forth in Table 1.

In a second aspect, the invention provides an isolated nucleic acid comprising a nucleotide sequence as set forth in SEQ ID NO:1, or a variant thereof.

In one embodiment, a variant nucleic acid of the second aspect comprises a nucleotide sequence selected from the group consisting of: SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; and SEQ ID NO:15.

In another embodiment, the variant has at 50%, 55%, 60%, 65%, 70%, 75%, 80%, 85% or 90% and more preferably at least 95%, 96%, 97%, 98% or 99% sequence identity to the isolated nucleic acid set forth in SEQ ID NO:1.

In a third aspect, the invention provides an isolated gene comprising the nucleotide sequence of the first aspect or the nucleotide sequence of the second aspect operably linked to a transcribable DNA sequence encoding SEQ ID NO: 28.

In a fourth aspect, the invention provides a chimeric gene comprising the isolated nucleic acid of the first aspect or the isolated nucleic acid of the second aspect operably linked to a heterologous nucleic acid.

In a fifth aspect, the invention provides a genetic construct comprising an isolated nucleic acid selected from the group consisting of: the isolated nucleic acid of the first aspect; the isolated nucleic acid of the second aspect; a transcribable DNA sequence encoding SEQ ID NO: 28; and the chimeric gene of the fourth aspect together with one or more other nucleotide sequences.

Advantageously, said one or more other nucleotide sequences includes but is not limited to, elements such as enhancers of transcription and translation, sequences for autonomous replication in prokaryotes, regulatory elements for mRNA processing, selectable markers and screenable markers.

In one preferred embodiment, the genetic construct is an expression vector comprising an isolated nucleic acid selected from the group consisting of: the isolated nucleic acid of the first aspect; and the isolated nucleic acid of the second aspect.

In another preferred embodiment, the genetic construct is an expression construct comprising the isolated gene of the third aspect or the chimeric gene of the fourth aspect.

The expression construct may be further characterized in that said isolated nucleic acid is capable of directing transcription preferentially in endosperm of wheat.

In a sixth aspect, the invention provides a host cell transformed with the genetic construct of the fifth aspect.

Suitably, the host cell is derived from a plant such as a cereal.

Preferably, the host cell is derived from a cereal which comprises at least an endosperm.

More preferably, the host cell is derived from wheat.

In a seventh aspect, the invention provides a method of producing a recombinant protein including the step of introducing into a plant host cell or tissue the genetic construct of the fifth aspect which is capable of producing said recombinant protein.

Preferably, the plant is a cereal such as, but not limited to, wheat.

In an eighth aspect, the invention provides a method of facilitating targeted expression to a plant endosperm including the step of expressing the chimeric gene of the fourth aspect in the endosperm of a plant.

Preferably, the plant is a cereal such as, but not limited to, wheat. More preferably, the cereal is wheat.

In a ninth aspect, the invention provides a genetically transformed plant comprising the isolated nucleic acid of the first or second aspect.

Plants encompass any taxonomic grouping thereof, including angiosperms, gymnosperms, monocotyledons and dicotyledons. Preferred plants are monocotyledons such as cereals, sugarcane, bananas and pineapples, but without limitation thereto.

More preferably, the plant is a cereal.

Even more preferably, the cereal is wheat.

Preferably, the transformed plant has an altered phenotype compared to a corresponding non-transformed plant.

Preferably, the altered phenotype results from expression of a heterologous nucleic acid.

The invention also provides cells, tissues, leaves, fruit, flowers, seeds and other reproductive material, material used for vegetative propagation, progeny plants including F1 hybrids, male-sterile plants and all other plants and plant products derived from genetically transformed plants of the invention.

Preferably, the plant product is a seed.

More preferably, the seed is a product of a cereal such as, but not limited to, wheat.

Even more preferably, the cereal is wheat.

In a tenth aspect, the invention provides an isolated polypeptide comprising an amino acid sequence as set forth in SEQ ID NO: 28.

In an eleventh aspect, the invention provides an antibody, or a fragment thereof that binds to the isolated polypeptide of the tenth aspect or a fragment thereof.

Throughout this specification, unless the context requires otherwise, the words “comprise”, “comprises” and “comprising” will be understood to imply the inclusion of a stated integer or group of integers but not the exclusion of any other integer or group of integers.

### BRIEF DESCRIPTION OF THE FIGURES

In order that the invention may be readily understood and put into practical effect, preferred embodiments will now be described by way of example with reference to the accompanying figures wherein like reference numerals refer to like parts and wherein:

FIG. 1: Relative abundance of TagA across all wheat (cv. Banks) LongSAGE libraries. All libraries were constructed from whole seed except for 14* which was constructed from pericarp tissue.

FIG. 2: Gel purification of genome-walking PCR products. Marker in left lane (SM) with band size indicated. First and second round PCR products present in lanes 1-4 and 5-8, respectively. Negative controls for each round marked with a dash (-). The band containing the correct promoter sequence is marked by an arrow.

FIG. 3: Alignment of 14 dpa developing wheat seed ESTs and Unigene cluster Ta.2025 EST sequences (gn1|UG|) that match TagA. Base positions identical to that of the first sequence are indicated by a stop (.) with sequence gaps marked by a dash (-). The longSAGE tag sequence (TAG) corresponding to TagA and primer sequences (PROM1_{—}1A, PROM1_{—}1B) for genome-walking are boxed. The putative coding region is in bold, with flanking start (ATG) and stop (TAG) codons underlined. The sequences may be identified as follows: TaBaD14EST-1e_G06 (SEQ ID NO: 16), TaBaD14EST-1f_G05 (SEQ ID NO: 17), TaBaD14EST-1-M_C_{—}05 (SEQ ID NO:18), TaBaD14EST-1d_F06 (SEQ ID NO: 19), TaBaD14EST-1f_C_{—}02 (SEQ ID NO: 20), TaBaD14EST-1B_E10 (SEQ ID NO:21), TaBaD14EST1g_B06 (SEQ ID NO: 22), TaBaD14EST-1d_E03 (SEQ ID NO: 23), TaBaD14EST-1d_D02 (SEQ ID NO: 24), TaBaD14EST-1-M_G02 (SEQ ID NO: 25), TaBaD14EST-1f_F02 (SEQ ID NO: 26) and TaBaD14EST-1f_F03 (SEQ ID NO: 27).

FIG. 4: Promoter sequence alignment with consensus sequence. Base positionds identical to the consensus are indicated by a stop (.) with gaps marked by a dash (-). Regions identical to EST sequences are highlighted. The sequences may be identified as follows: Consensus (SEQ ID NO: 1), Prom1_{—}2_{—}17 (SEQ ID NO: 2), Prom1_{—}2_{—}6 (SEQ ID NO: 3), Prom1_{—}2_{—}3 (SEQ ID NO: 4), Prom1_{—}2_{—}13 (SEQ ID NO: 5), Prom1_{—}2_{—}18 (SEQ ID NO: 6), Prom1_{—}2_{—}7 (SEQ ID NO: 7), Prom1_{—}2_{—}15 (SEQ ID NO: 8), Prom1_{—}2_{—}16 (SEQ ID NO: 9), Prom1_{—}2_{—}21 (SEQ ID NO: 10), Prom1_{—}2_{—}19 (SEQ ID NO: 11), Prom1_{—}2_{—}20 (SEQ ID NO: 12), Prom1_{—}2_{—}24 (SEQ ID NO: 13), Prom1_{—}2_{—}14 (SEQ ID NO: 14), Prom1_{—}2_{—}10 (SEQ ID NO: 15). The corresponding promoter sequence from cultivar cv Banks genomic DNA indicates that the sequence is identical to Bob White sequence (SEQ ID NO: 1) from position 100-1354 of SEQ ID NO:1 (SEQ ID NO:29).

FIG. 5: Putative amino acid sequence translation of ESTs matching TagA (SEQ ID NO: 28), with selected PredictProtein motif and structure prediction details.

### DETAILED DESCRIPTION OF THE INVENTION

The promoter regions and sequences identified as part of the present invention provide new and advantageous tools for development of a recombinant protein expression system in cereal plants, and in particular, in wheat. The suitability of these promoters for this purpose results from their ability to direct high levels of transcription in the endosperm of developing wheat seed. The cereal endosperm is an ideal candidate as a compartment for overexpression of recombinant proteins as it is the natural site of storage protein accumulation and provides a stable environment for recombinant protein synthesis.

Transcription of DNA into RNA by DNA-dependent RNA polymerases is a highly complex and tightly regulated process owing to the need to ensure correct temporal and spatial expression of many thousands of genes. Transcriptional control elements are generally located adjacent to and/or upstream of a transcribable DNA sequence and provide a road map for the regulation of transcription and further gene expression events. With an ability to direct accurate transcription initiation, promoter regions represent the primary transcriptional control element.

Therefore in a broad aspect, the invention provides an isolated nucleic acid comprising a nucleotide sequence which corresponds to a promoter-active region isolated adjacent to the start of a transcribable DNA sequence.

In one particular aspect, the invention provides an isolated nucleic acid comprising a nucleotide sequence that corresponds to a promoter-active region of a gene comprising a transcribable DNA sequence encoding SEQ ID NO: 28.

The terms “promoter”, “promoter region” and “promoter-active region” refer to a nucleotide sequence which directs expression of a transcribable DNA sequence to which it is operably linked, by initiating, regulating or otherwise controlling transcription of said transcribable DNA sequence.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native or recombinant form.

The term “nucleic acid” as used herein designates single- or double-stranded mRNA, RNA, cRNA and DNA inclusive of cDNA, genomic DNA and DNA-RNA hybrids.

By “operably linked” is meant functionally linked. By way of example only, a promoter nucleic acid of the invention is operably linked to a transcribable nucleic acid so as to be capable of initiating, controlling, regulating or otherwise directing transcription of the transcribable nucleic acid.

The term “transcribable DNA sequence” or “transcribed DNA sequence”, excludes a promoter region that drives transcription. Depending on the aspect of the invention, the transcribable sequence may be derived in whole or in part from any source known to the art, including a plant, a fungus, an animal, a bacterial genome or episome, eukaryotic, nuclear or plasmid DNA, cDNA, viral DNA or chemically synthesized DNA. A transcribable sequence may contain one or more modifications in either the coding or the untranslated regions which could affect the biological activity or the chemical structure of the expression product, the rate of expression or the manner of expression control. Such modifications include, but are not limited to, insertions, deletions and substitutions of one or more nucleotides. The transcribable sequence may contain an uninterrupted coding sequence or it may include one or more introns, bound by the appropriate splice junctions. The transcribable sequence may also encode a fusion protein. It is contemplated that introduction into plant tissue of chimeric nucleic acid constructs of the invention will include constructions wherein the transcribable sequence and its promoter are each derived from different species.

In order to sustain the intricate nature of gene expression in eukaryotes, promoter regions have evolved as highly diverse entities in both structure and function. For instance, a promoter may be categorised as either ‘strong’ or ‘weak’, which are generic terms that refer to the relative levels of transcript production. Moreover, certain promoters are capable of directing RNA production in many or all tissues and are thus termed “constitutive promoters”. Alternatively, other promoters have been shown to direct RNA production at higher levels only in particular types of cells or tissues and are referred to as “tissue-specific promoters”.

The promoter region of the present invention is highly active in the endosperm of a cereal including barley, corn, wheat, maize and the like but is not limited thereto.

Preferably, the cereal is wheat. Suitably, a Bob White or Banks variety of wheat may be used.

Regardless of the type of promoter, a promoter-active region of a transcribable DNA sequence must be of a sufficient length such that it is capable of initiating and regulating transcription of a DNA sequence to which it is coupled. The promoter region may range in length from anywhere between 100 bp to several kilobases.

Preferably, the promoter-active region is between 100 bp and 4 kb. More preferably, the promoter-active region is greater than 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1000 bp, 1100 bp, 1200 bp, 1300 bp and 1400 bp. Even more preferably, the promoter-active region is greater than 1500 bp in length. Preferably, the promoter-active region is less than 4 kb, 3.5 kb, 3 kb, 2.5 kb and 2 kb. In certain forms of these embodiments, although not necessarily the only form, the promoter-active region corresponds to a region about 100 bp to about 400 bp upstream of the translation start site of a transcribable DNA sequence such as, but not limited to, SEQ ID NO: 28.

In a preferred embodiment, the promoter-active region comprises a nucleotide sequence as set forth in SEQ ID NO: 1, or a variant thereof.

The term “variant” is used in the context of a nucleic acid which displays a definable level of sequence identity with a reference nucleic acid. For example, a variant nucleic acid is hybridizable with a reference sequence under stringent conditions that are defined hereinafter, or shares a percent level of sequence identity definable using a sequence comparison algorithm as hereinafter described. Variants also encompass nucleic acids in which one or more nucleotides have been added or deleted, or replaced with different nucleotides or modified bases (eg. inosine, methylcytosine). In this regard, it is well understood in the art that certain alterations inclusive of mutations, additions, deletions and substitutions can be made to a reference nucleic acid whereby the altered nucleotide sequence retains the biological function or activity of the reference nucleotide sequence. The term “variant” also include naturally occurring allelic variants.

Variants of an earlier prepared variant or non-variant version of an isolated natural promoter according to the invention can be artificially engineered using an assortment of recombinant techniques. Non-limiting examples of suitable techniques include random mutagenesis (e.g., transposon mutagenesis), oligonucleotide-mediated (or site-directed) mutagenesis, PCR mutagenesis and cassette mutagenesis.

Therefore in one embodiment, the invention provides a variant nucleic acid which is a variant of the nucleotide sequence set forth in SEQ ID NO: 1. In certain embodiments, said variant may comprise a nucleotide sequence selected from the group consisting of: SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; and SEQ ID NO:15. In other particular embodiments, the variant nucleic acid may comprise any one or more of the variant residues as set forth in SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; and SEQ ID NO:15.

In other certain embodiments, the variant nucleic acid may comprise a nucleotide sequence which corresponds to a promoter sequence derived from another cultivar of wheat. In particular embodiments, the variant nucleic acid may comprise a nucleotide sequence from cultivar cv Banks genomic DNA which is a nucleotide sequence that is identical to the nucleotide sequence from position 100-1354 of SEQ ID NO: 1 (SEQ ID NO:29).

The invention also contemplates variants of the promoter-active region or isolated promoter sequence that share a relationship based upon homology between sequences.

“Homology” refers to the percentage number of nucleotides of a nucleotide sequence that are identical to a reference nucleotide sequence. Homology may be determined using sequence comparison programs such as BESTFIT (Deveraux et al. 1984, Nucleic Acids Research 12, 387-395) which is incorporated herein by reference. In this way sequences of a similar or substantially different length to those cited herein might be compared by insertion of gaps into the alignment, such gaps being determined, for example, by the comparison algorithm used by BESTFIT.

Terms used to describe sequence relationships between two or more nucleotide sequences include “reference sequence”, “comparison window”, “sequence identity”, “percentage of sequence identity” and “substantial identity”. A “reference sequence” is at least 6 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 6 to 12 contiguous residues that is compared to a reference sequence. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA, incorporated herein by reference) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389, which is incorporated herein by reference. A detailed discussion of sequence analysis can be found in Unit 19.3 of Ausubel et al., “Current Protocols in Molecular Biology”, John Wiley & Sons Inc, 1994-1998, Chapter 15.

The term “sequence identity” as used herein refers to the extent that sequences are identical on a nucleotide-by-nucleotide basis over a window of comparison. Thus, a “percentage of sequence identity” is calculated by comparing two optimally aligned sequences over the window of comparison, determining the number of positions at which the identical nucleic acid base (e.g., A, T, C, G, I) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software, which is incorporated herein by reference.

In one embodiment, nucleic acid variants share at least 50%, 55%, 60%, 65%, 70%, 75%, 80%, 85% or 90% and more preferably at least 95%, 96%, 97%, 98% or 99% sequence identity with the isolated nucleic acids of the invention.

In another embodiment, nucleic acid variants hybridise to nucleic acids of the invention, including fragments, under at least low stringency conditions, preferably under at least medium stringency conditions and more preferably under high stringency conditions.

“Hybridise and Hybridisation” is used herein to denote the pairing of at least partly complementary nucleotide sequences to produce a DNA-DNA, RNA-RNA or DNA-RNA hybrid. Hybrid sequences comprising complementary nucleotide sequences occur through base-pairing.

Modified purines (for example, inosine, methylinosine and methyladenosine) and modified pyrimidines (thiouridine and methylcytosine) may also engage in base pairing.

“Stringency” as used herein, refers to temperature and ionic strength conditions, and presence or absence of certain organic solvents and/or detergents during hybridisation. The higher the stringency, the higher will be the required level of complementarity between hybridizing nucleotide sequences.

“Stringent conditions” designates those conditions under which only nucleic acid having a high frequency of complementary bases will hybridize.

Reference herein to low stringency conditions includes and encompasses:—
(i) from at least about 1% v/v to at least about 15% v/v formamide and from at least about 1 M to at least about 2 M salt for hybridisation at 42° C., and at least about 1 M to at least about 2 M salt for washing at 42° C.; and
(ii) 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature.

Medium stringency conditions include and encompass:—
(i) from at least about 16% v/v to at least about 30% v/v formamide and from at least about 0.5 M to at least about 0.9 M salt for hybridisation at 42° C., and at least about 0.5 M to at least about 0.9 M salt for washing at 42° C.; and
(ii) 1% Bovine Serum Albumin (BSA), 1 mM EDTA, 0.5 M NaHPO₄ (pH 7.2), 7% SDS for hybridization at 65° C. and (a) 2×SSC, 0.1% SDS; or (b) 0.5% BSA, 1 mM EDTA, 40 mM NaHPO₄ (pH 7.2), 5% SDS for washing at 42° C.

High stringency conditions include and encompass:—
(i) from at least about 31% v/v to at least about 50% v/v formamide and from at least about 0.01 M to at least about 0.15 M salt for hybridisation at 42° C., and at least about 0.01 M to at least about 0.15 M salt for washing at 42° C.;
(ii) 1% BSA, 1 mM EDTA, 0.5 M NaHPO₄ (pH 7.2), 7% SDS for hybridization at 65° C., and (a) 0.1×SSC, 0.1% SDS; or (b) 0.5% BSA, 1 mM EDTA, 40 mM NaHPO₄ (pH 7.2), 1% SDS for washing at a temperature in excess of 65° C. for about one hour; and
(iii) 0.2×SSC, 0.1% SDS for washing at or above 68° C. for about 20 minutes.

In general, the Tₘ of a duplex DNA decreases by about 1° C. with every increase of 1% in the number of mismatched bases.

Notwithstanding the above, stringent conditions are well known in the art, such as described in Chapters 2.9 and 2.10 of Ausubel et al., supra, which are herein incorporated by reference. A skilled addressee will also recognize that various factors can be manipulated to optimize the specificity of the hybridization. Optimization of the stringency of the final washes can serve to ensure a high degree of hybridization.

Typically, complementary nucleotide sequences are identified by blotting techniques that include a step whereby nucleotides are immobilized on a matrix (preferably a synthetic membrane such as nitrocellulose), a hybridization step, and a detection step. Southern blotting is used to identify a complementary DNA sequence; Northern blotting is used to identify a complementary RNA sequence. Dot blotting and slot blotting can be used to identify complementary DNA/DNA, DNA/RNA or RNA/RNA polynucleotide sequences. Such techniques are well known by those skilled in the art, and have been described in Ausubel et al., supra, at pages 2.9.1 through 2.9.20, herein incorporated by reference.

Nucleic acid variants of the invention may be prepared according to the following procedure:
(i) obtaining a nucleic acid extract from a suitable host, for example a bacterial species;
(ii) creating primers which are optionally degenerate wherein each comprises a fragment of a nucleotide sequence which corresponds to a transcribable DNA sequence encoding SEQ ID NO: 28 or alternatively, a nucleotide sequence adjacent to transcribable DNA sequence encoding SEQ ID NO: 28; and
(iii) using said primers to amplify, via nucleic acid amplification techniques, one or more amplification products from said nucleic acid extract.

As used herein, an “amplification product” refers to a nucleic acid product generated by nucleic acid amplification techniques.

As used herein, a “nucleic acid sequence amplification technique” includes but is not limited to polymerase chain reaction (PCR) as for example described in Chapter 15 of CURRENT PROTOCOLS 1N MOLECULAR BIOLOGY Eds. Ausubel et al. (John Wiley & Sons NY USA 1995-2001) strand displacement amplification (SDA); rolling circle replication (RCR) as for example described in International Application WO 92/01813 and International Application WO 97/19193; nucleic acid sequence-based amplification (NASBA) as for example described by Sooknanan et al. 1994, Biotechniques 17 1077; ligase chain reaction (LCR) as for example described in International Application WO89/09385 and Chapter 15 of CURRENT PROTOCOLS IN MOLECULAR BIOLOGY supra; Q-β replicase amplification as for example described by Tyagi et al. 1996, Proc. Natl. Acad. Sci. USA 93: 5395 and helicase-dependent amplification as for example described in International Publication WO 2004/02025.

A person skilled in the art will readily appreciate that the invention also contemplates a promoter-active fragment of the promoter-active region as well as nucleotide sequence variants thereof. It will be readily understood that a promoter-active fragment of a promoter sequence, when fused to a particular gene and introduced into a plant cell, causes expression of the gene at a higher level than is possible in the absence of such fragment. The activity of a promoter can be determined by methods well known in the art. For example, reference may be made to Medberry et al. (1992, Plant Cell 4:185; 1993, The Plant J. 3:619, incorporated herein by reference), Sambrook et al. (1989, supra) and McPherson et al. (U.S. Pat. No. 5,164,316, incorporated herein by reference).

Certain minimal nucleic acid regions, otherwise known as regulatory elements, are required for a fragment to possess promoter-activity. Such control elements are a mixture of distinct promoter sequence elements such as but not limited to, the TATA box, the INR element, the BRE element, the plastid element and the endosperm specific element as well as binding sites for gene-specific transcription factors. It is well known in the art that for example, the TATA box and the INR element are able to independently initiate accurate transcription.

Preferably, the promoter-active fragment comprises at least one element from the list set forth in Table 1.

More preferably, the promoter-active fragment comprises at least two elements from the list set forth in Table 1.

Table 2 provides a more exhaustive list of possible regulatory gene elements located in the promoter-active fragment.

Transcribable DNA sequences and Proteins

The promoters of the present invention were initially localised by their ability to direct high levels of production of a transcribed DNA sequence in the endosperm of developing wheat seed. Serial analysis of gene expression (SAGE), as described hereinafter, was used to identify the transcribable DNA sequences of the present invention. Exemplary nucleotide sequences of this type are set forth in SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26 and SEQ ID NO: 27.

However, it will be understood that the present invention is not restricted to use of any particular method for identifying such differentially expressed genes. For example, alternative procedures for identifying gene expressed differentially in various tissues include, but are not restricted to: cDNA and genomic subtractive hybridisation as for example described by Bulman and Neill (1996, In “Plant Gene Isolation: Principles and Practice”, G. D. Foster and D. Twell, eds Chichester, UK, Wiley, pp 369-397); multi-probe fluorescent analysis of microscopic cDNA arrays as for example described by Schena (1996 BioEssays 18:427-431); mRNA differential display as for example described by Liang and Pardee (1992, Science 257:967-970) and by Callard et al. (1994, BioTechniques 16:1096-1103); computer analysis of mRNA abundance based on frequency of occurrence of identical sequences emerging from large-scale sequencing of cDNA ends (ESTs) as for example taught by Cooke et al (1996, EST and genomic sequencing projects. In Plant Gene Isolation: Principles and Practice, supra, pp. 410-419); or promoter tagging by insertional mutagenesis with promoterless reporter genes as for example disclosed by Lindsey and Topping (1996, T-DNA-mediated insertional mutagenesis. In Plant Gene Isolation: Principles and Practice, supra, pp. 275-300) and Mudge and Birch (1998, Austral. J. Plant Physiol. 25:637-643), which are all incorporated herein by reference.

It will also be appreciated that the invention contemplates isolated nucleic acid variants of the transcribable DNA sequence set forth in SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26 and SEQ ID NO: 27.

In one embodiment, nucleic acid variants share at least 50%, 55%, 60%, 65%, 70%, 75%, 80%, 85% or 90% and more preferably at least 95%, 96%, 97%, 98% or 99% sequence identity with the isolated nucleic acids of the invention.

As hereinbefore described, a transcribable DNA sequence contains, inter alia, nucleotide sequence which encodes an amino acid sequence of a protein. The transcribable DNA sequences of the present invention encode an amino acid sequence as set forth in SEQ ID NO: 28. Although not wishing to be bound by any particular theory, secondary structure prediction analysis suggests that this amino acid sequence corresponds to a microbody-associated protein

By “protein” is meant an amino acid polymer. The amino acids may be natural or non-natural amino acids, D- or L-amino acids as are well understood in the art.

The term “protein” includes and encompasses “peptide”, which is typically used to describe a protein having no more than fifty (50) amino acids and “polypeptide”, which is typically used to describe a protein having more than fifty (50) amino acids.

### Genes, Chimeric Genes and Genetic Constructs

The isolated promoter, or a variant thereof, of the present invention may be fused to either to a nucleic acid to which it is naturally associated or a heterologous nucleic acid to form a gene and chimeric gene respectively. The promoter of the present invention provides an added advantage of high levels of transcript production.

By “heterologous nucleic acid” is meant a nucleic acid distinct from an isolated promoter of the invention. Operationally, the heterologous nucleic acid is operably linked to an isolated promoter nucleic acid of the invention to achieve expression of the heterologous nucleic acid. The term heterologous nucleic acid encompasses transcribable DNA as defined hereinbefore.

The term “gene” is used herein to describe a discrete nucleic acid locus, unit or region within a genome that may comprise one or more of introns, exons, splice sites, open reading frames and 5′ and/or 3′ non-coding regulatory sequences such as a promoter and/or a polyadenylation sequence. “Gene” also encompasses an RNA copy or cDNA copy of the gene.

“Chimeric gene” is defined herein as a nucleic acid, preferably a DNA molecule, either single- or double-stranded, which includes an isolated nucleic acid of the invention, variant or promoter-active fragment, operably linked to a heterologous nucleic acid.

Therefore in one embodiment, the invention contemplates an isolated gene comprising a promoter-active region operably linked to a transcribable DNA sequence. In a preferred form of this embodiment, the transcribable DNA sequence is located adjacent to the promoter-active region in vivo. More preferably, the transcribable DNA sequence encodes SEQ ID NO: 28.

It is also envisaged that the invention provides a chimeric gene for the purpose of transformation and expression of a heterologous nucleic acid. It is readily contemplated that the invention is suited to expression of a broad spectrum of molecules encoded by said heterologous nucleic acid. However, the invention is particularly suited to expression of a heterologous nucleic acid which encodes a biologically-active protein for use as a biopharmaceutical. Typically, although not exclusively, said biologically-active protein will possess palliative properties. Non-limiting examples include growth factors, kinases, immunostimulatory antigens, antibodies, regulatory proteins such as cytokines, chemokines and the like.

The promoters and the isolated genes of the present invention are quite amenable for inclusion into a genetic construct. It can be readily appreciated by a person skilled in the art that a genetic construct is a nucleic acid comprising any one of a number of nucleotide sequence elements, the function of which depends upon the desired use of the construct. Uses range from vectors for the general manipulation and propagation of recombinant DNA to more complicated applications such as prokaryotic or eukaryotic expression of a heterologous nucleic acid and production of genetically-modified plants or animals. Typically, although not exclusively, genetic constructs are designed to provide more than one application. By way of example only, a genetic construct whose intended end use is recombinant protein expression in a eukaryotic system may have incorporated nucleotide sequences for such functions as cloning and propagation in prokaryotes over and above sequences required for expression. An important consideration when designing and preparing such genetic constructs are the required nucleotide sequences for the intended application.

In view of the foregoing, it is evident to a person of skill in the art that genetic constructs are versatile tools that can be adapted for any one of a number of purposes.

Therefore in one particular aspect, the invention provides a variety of genetic constructs comprising the isolated nucleic acids of the invention together with one or more other nucleotide sequences.

In one preferred embodiment, the genetic construct is an expression construct comprising a nucleotide sequence which corresponds to a promoter-active region of the present invention together with one or more other nucleotide sequences. In one form of this embodiment, the promoter-active region is of a gene comprising a transcribable DNA sequence SEQ ID NO: 28. In another form of this particular embodiment, the nucleotide sequence which corresponds to a promoter is set forth in SEQ ID NO: 1 or a variant thereof.

In another preferred embodiment, the genetic construct is an expression vector which is designed to receive an isolated nucleic acid comprising a nucleotide sequence which encodes a protein for recombinant expression. Preferably, the expression vector comprises at least a promoter and in addition, one or more other nucleotide sequences which are required for manipulation, propagation and expression of recombinant DNA.

In one form of this embodiment, the promoter is a promoter-active region of a gene comprising a transcribable DNA sequence. Preferably, the transcribable DNA sequence encodes SEQ ID NO: 28.

In another form of this embodiment, the nucleotide sequence which corresponds to a promoter is set forth in SEQ ID NO: 1 or a variant thereof.

By “vector” is meant a nucleic acid, preferably a DNA molecule derived, for example, from a plasmid, bacteriophage, or plant virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integratable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art.

In another preferred embodiment, the genetic construct comprises an isolated nucleic acid comprising a nucleotide sequence which corresponds to an expressible sequence.

Preferably, the expressible sequence is an isolated gene or chimeric gene of the present invention.

More preferably, the expressible sequence is a chimeric gene of the present invention.

### Additional Sequences

The genetic construct of the present invention can further include enhancers, either translation or transcription enhancers, as may be required. These enhancer regions are well known to persons skilled in the art, and can include the ATG initiation codon and adjacent sequences. The initiation codon must be in phase with the reading frame of the coding sequence relating to the heterologous or endogenous DNA sequence to ensure translation of the entire sequence. The translation control signals and initiation codons can be of a variety of origins, both natural and synthetic. Translational initiation regions may be provided from the source of the transcriptional initiation region, or from the heterologous or endogenous DNA sequence. The sequence can also be derived from the source of the promoter selected to drive transcription, and can be specifically modified so as to increase translation of the mRNA.

Examples of transcriptional enhancers include, but are not restricted to, elements from the CaMV 35S promoter and octopine synthase genes as for example described by Last et al. (U.S. Pat. No. 5,290,924, which is incorporated herein by reference). It is proposed that the use of an enhancer element such as the ocs element, and particularly multiple copies of the element, will act to increase the level of transcription from adjacent promoters when applied in the context of plant transformation.

As the DNA sequence inserted between the transcription initiation site and the start of the coding sequence, i.e., the untranslated leader sequence, can influence gene expression, one can also employ a particular leader sequence. Preferred leader sequences include those that comprise sequences selected to direct optimum expression of the heterologous or endogenous DNA sequence. For example, such leader sequences include a preferred consensus sequence which can increase or maintain mRNA stability and prevent inappropriate initiation of translation as for example described by Joshi (1987, Nucl. Acid Res., 15:6643), which is incorporated herein by reference. However, other leader sequences, e.g., the leader sequence of RTBV, have a high degree of secondary structure that is expected to decrease mRNA stability and/or decrease translation of the mRNA. Thus, leader sequences (i) that do not have a high degree of secondary structure, (ii) that have a high degree of secondary structure where the secondary structure does not inhibit mRNA stability and/or decrease translation, or (iii) that are derived from genes that are highly expressed in plants, will be most preferred.

Regulatory elements such as the sucrose synthase intron as, for example, described by Vasil et al. (1989, Plant Physiol., 91:5175), the Adh intron I as, for example, described by Canis et al. (1987, Genes Develop., II), or the TMV omega element as, for example, described by Gallie et al. (1989, The Plant Cell, 1:301) can also be included where desired. Other such regulatory elements useful in the practice of the invention are known to those of skill in the art.

Additionally, targeting sequences may be employed to target a protein product of the heterologous or endogenous nucleotide sequence to an intracellular compartment within plant cells or to the extracellular environment. For example, a DNA sequence encoding a transit or signal peptide sequence may be operably linked to a sequence encoding a desired protein such that, when translated, the transit or signal peptide can transport the protein to a particular intracellular or extracellular destination, respectively, and can then be post-translationally removed. Transit or signal peptides act by facilitating the transport of proteins through intracellular membranes, e.g., vacuole, vesicle, plastid and mitochondrial membranes, whereas signal peptides direct proteins through the extracellular membrane. For example, the transit or signal peptide can direct a desired protein to a particular organelle such as a plastid (e.g., a chloroplast), rather than to the cytoplasm. Thus, the genetic construct can further comprise a plastid transit peptide encoding DNA sequence operably linked between a promoter region or promoter variant according to the invention and the heterologous or endogenous nucleotide sequence. For example, reference may be made to Heijne et al. (1989, Eur. J. Biochem., 180:535) and Keegstra et al. (1989, Ann. Rev. Plant Physiol. Plant Mol. Biol., 40:471), which are incorporated herein by reference.

An isolated nucleic acid of the present invention can also be introduced into a vector, such as a plasmid. Plasmid vectors include additional DNA sequences that provide for easy selection, amplification, and transformation of the expression cassette in prokaryotic and eukaryotic cells, e.g., pUC-derived vectors, pSK-derived vectors, pGEM-derived vectors, pSP-derived vectors, or pBS-derived vectors. Additional DNA sequences include origins of replication to provide for autonomous replication of the vector, selectable marker genes, preferably encoding antibiotic or herbicide resistance, unique multiple cloning sites providing for multiple sites to insert DNA sequences or genes encoded in the chimeric DNA construct, and sequences that enhance transformation of prokaryotic and eukaryotic cells.

The vector preferably contains an element(s) that permits stable integration of the vector into the host cell genome or autonomous replication of the vector in the cell independent of the genome of the cell. The vector may be integrated into the host cell genome when introduced into a host cell. For integration, the vector may rely on the heterologous or endogenous DNA sequence or any other element of the vector for stable integration of the vector into the genome by homologous recombination. Alternatively, the vector may contain additional nucleic acid sequences for directing integration by homologous recombination into the genome of the host cell. The additional nucleic acid sequences enable the vector to be integrated into the host cell genome at a precise location in the chromosome. To increase the likelihood of integration at a precise location, the integrational elements should preferably contain a sufficient number of nucleic acids, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences.

For autonomous replication, the vector may further comprise an origin of replication enabling the vector to replicate autonomously in the host cell in question. Examples of bacterial origins of replication are the origins of replication of plasmids pBR322, pUC19, pACYC 177, and pACYC 184 permitting replication in E. coli, and pUB110, pE194, pTA1060, and pAM.beta.1 permitting replication in Bacillus. The origin of replication may be one having a mutation to make its function temperature-sensitive in a Bacillus cell (see, e.g., Ehrlich, 1978, Proc. Natl. Acad. Sci. USA 75:1433).

### Marker Genes

To facilitate identification of transformants, the genetic construct desirably comprises a selectable or screenable marker gene as, or in addition to, the expressible heterologous or endogenous nucleotide sequence. The actual choice of a marker is not crucial as long as it is functional (i.e., selective) in combination with the plant cells of choice. The marker gene and the heterologous or endogenous nucleotide sequence of interest do not have to be linked, since co-transformation of unlinked genes as, for example, described in U.S. Pat. No. 4,399,216 is also an efficient process in plant transformation.

Included within the terms selectable or screenable marker genes are genes that encode a “secretable marker” whose secretion can be detected as a means of identifying or selecting for transformed cells. Examples include markers that encode a secretable antigen that can be identified by antibody interaction, or secretable enzymes that can be detected by their catalytic activity. Secretable proteins include, but are not restricted to, proteins that are inserted or trapped in the cell wall (e.g., proteins that include a leader sequence such as that found in the expression unit of extensin or tobacco PR-S); small, diffusible proteins detectable, e.g. by ELISA; and small active enzymes detectable in extracellular solution (e.g., α-amylase, β-lactamase, phosphinothricin acetyltransferase).

### Selectable Markers

Examples of bacterial selectable markers are the dal genes from Bacillus subtilis or Bacillus licheniformis, or markers that confer antibiotic resistance such as ampicillin, kanamycin, erythromycin, chloramphenicol or tetracycline resistance. Exemplary selectable markers for selection of plant transformants include, but are not limited to, a hyg gene which encodes hygromycin B resistance; a neomycin phosphotransferase (neo) gene conferring resistance to kanamycin, paromomycin, G418 and the like as, for example, described by Potrykus et al. (1985, Mol. Gen. Genet. 199:183); a glutathione-S-transferase gene from rat liver conferring resistance to glutathione derived herbicides as, for example, described in EP-A 256 223; a glutamine synthetase gene conferring, upon overexpression, resistance to glutamine synthetase inhibitors such as phosphinothricin as, for example, described WO87/05327, an acetyl transferase gene from Streptomyces viridochromogenes conferring resistance to the selective agent phosphinothricin as, for example, described in EP-A 275 957, a gene encoding a 5-enolshikimate-3-phosphate synthase (EPSPS) conferring tolerance to N-phosphonomethylglycine as, for example, described by Hinchee et al. (1988, Biotech., 6:915), a bar gene conferring resistance against bialaphos as, for example, described in WO91/02071; a nitrilase gene such as bxn from Klebsiella ozaenae which confers resistance to bromoxynil (Stalker et al., 1988, Science, 242:419); a dihydrofolate reductase (DHFR) gene conferring resistance to methotrexate (Thillet et al., 1988, J. Biol. Chem., 263:12500); a mutant acetolactate synthase gene (ALS), which confers resistance to imidazolinone, sulfonylurea or other ALS-inhibiting chemicals (EP-A-154 204); a mutated anthranilate synthase gene that confers resistance to 5-methyl tryptophan; or a dalapon dehalogenase gene that confers resistance to the herbicide.

### Screenable Markers

Preferred screenable markers include, but are not limited to, a uidA gene encoding a β-glucuronidase (GUS) enzyme for which various chromogenic substrates are known; a β-galactosidase gene encoding an enzyme for which chromogenic substrates are known; an aequorin gene (Prasher et al., 1985, Biochem. Biophys. Res. Comm., 126:1259), which may be employed in calcium-sensitive bioluminescence detection; a green fluorescent protein gene (Niedz et al., 1995 Plant Cell Reports, 14:403); a luciferase (luc) gene (Ow et al., 1986, Science, 234:856), which allows for bioluminescence detection; a β-lactamase gene (Sutcliffe, 1978, Proc. Natl. Acad. Sci. USA 75:3737), which encodes an enzyme for which various chromogenic substrates are known (e.g., PADAC, a chromogenic cephalosporin); an R-locus gene, encoding a product that regulates the production of anthocyanin pigments (red color) in plant tissues (Dellaporta et al., 1988, in Chromosome Structure and Function, pp. 263-282); an α-amylase gene (Ikuta et al., 1990, Biotech., 8:241); a tyrosinase gene (Katz et al., 1983, J. Gen. Microbiol., 129:2703) which encodes an enzyme capable of oxidizing tyrosine to dopa and dopaquinone which in turn condenses to form the easily detectable compound melanin; or a xylE gene (Zukowsky et al., 1983, Proc. Natl. Acad. Sci. USA 80:1101), which encodes a catechol dioxygenase that can convert chromogenic catechols.

### Genetically Modified Plants and Methods of Use

One broad application of the genetic constructs provided for by the present invention is a system for overexpression of recombinant proteins. It is envisaged that such a system is suitable for expression of any class of proteins including antibodies, technical proteins (such as commercially-available enzymes) and biologically-active proteins. However, the isolated nucleic acids and genetic constructs of the invention are particularly suited to a recombinant protein expression system based upon generation of a genetically-modified plant for the purpose of molecular farming.

The term “genetically-modified” broadly refers to introduction of a heterologous nucleic acid into a plant or animal. The heterologous nucleic acid may subsist in the organism by means of chromosomal integration into the host genome or alternatively, by episomal replication. Genetic-modification may, although not necessarily, result in alteration of the host organism phenotype.

By “molecular farming” is meant the use of genetically-enhanced plants for the production of biologically-active proteins for use as biopharmaceuticals. Twyman et al 2003 (Trends in Biotechnology, 21: 570-578) provides an example of host systems and expression technology which are useful in this technique and is incorporated herein by reference. The use of plants as a recombinant protein expression system provides a number of significant advantages over prokaryote-, yeast- and animal-based systems including low production costs, safety benefits due to absence of human pathogens, scalability and the ability to fold and assemble complex proteins accurately. A plethora of different platforms have been used for molecular farming including leafy crops, cereal and legume seeds, oilseeds, fruits, vegetables, hydroponic systems, algae and moss. A particular advantage conferred by seed-based production platforms is the ability of the seed to store proteins in a stable form for long periods of time. Moreover because high levels of recombinant protein accumulate in a small volume in the seed, effectively the starting material is a concentrated protein solution, which greatly facilitates purification and processing. Seeds from cereal crops are particularly advantageous because of high biomass yield and absence in seeds of problematic phenolic substances. Wheat provides an highly attractive expression host due to its low producer costs.

A desirable property of seed-based recombinant expression systems is the possibility of tissue-specific expression. Different compartments of a seed have specialised storage capacities. Typically, although not exclusively, the storage function in a seed is either divided between the embryo and the endosperm or assumed by cotyledons. The endosperm is a dedicated storage tissue with the sole purpose of accumulating nutrients for the germinating embryo and as such is an ideal compartment for recombinant protein expression. An added benefit of expression in a storage tissue is avoidance of heterologous protein accumulation in vegetative organs thus preventing toxicity to the host plant. As tissue-specific expression is directed by specific promoters, use of appropriate promoters in genetic constructs represents an important aspect of molecular farming.

Therefore in one particular aspect, the invention resides in a method of producing a recombinant protein which includes the step of introducing into a host cell or tissue the promoters and genetic constructs of the present invention.

In light of the foregoing, it is evident to a person of skill in the art that in another particular aspect, the invention provides a method of facilitating tissue-specific expression of a recombinant protein in a plant endosperm including the step of expressing a chimeric gene of the invention.

A recombinant protein may be conveniently prepared by a person skilled in the art using standard protocols as for example described in Sambrook et al., MOLECULAR CLONING. A Laboratory Manual (Cold Spring Harbor Press, 1989), incorporated herein by reference, in particular Sections 16 and 17; CURRENT PROTOCOLS IN MOLECULAR BIOLOGY Eds. Ausubel et al., (John Wiley & Sons, Inc. 1995-1999), incorporated herein by reference, in particular Chapters 10 and 16; and CURRENT PROTOCOLS IN PROTEIN SCIENCE Eds. Coligan et al., (John Wiley & Sons, Inc. 1995-1999) which is incorporated by reference herein, in particular Chapters 1, 5 and 6.

Suitable host cells for recombinant protein expression are plant cells and/or plant tissue.

Preferably, the host cell or tissue is derived from a plant such as, but not limited to, cereals, leafy crops, legumes, fruits and vegetables.

More preferably, the host cell or tissue is derived from a cereal.

The promoters and genetic constructs of the present invention are suited to generation of any type of genetically-modified plant which produces a seed during its life cycle. Non-limiting examples include cereals, legumes and leafy crops such as tobacco.

Preferably, the host cell is derived from a cereal such as, but not limited to, maize, rice, barley or wheat.

More preferably, the host cell is derived from wheat.

### Plant Transformation

The initial step in production of a genetically-modified plant is introduction of DNA into a plant host cell. A number of techniques are available for the introduction of DNA into a plant host cell. There are many plant transformation techniques well known to workers in the art, and new techniques are continually becoming known. The particular choice of a transformation technology will be determined by its efficiency to transform certain plant species as well as the experience and preference of the person practising the invention with a particular methodology of choice. It will be apparent to the skilled person that the particular choice of a transformation system to introduce a genetic construct into plant cells is not essential to or a limitation of the invention, provided it achieves an acceptable level of nucleic acid transfer. Guidance in the practical implementation of transformation systems for plant improvement is provided by Birch (1997, Annu. Rev. Plant Physiol. Plant Molec. Biol. 48: 297-326), which is incorporated herein by reference.

The term “transformation” means alteration of genotype by introduction of genetic material into an organism.

In principle both dicotyledonous and monocotyledonous plants that are amenable to transformation, can be modified by introducing a chimeric DNA construct according to the invention into a recipient cell and growing a new plant that harbors and expresses the heterologous or endogenous nucleotide sequence.

It will be appreciated that a variety of plant tissues can undergo transformation including leaf spindle or whorl, leaf blade, axillary buds, stems, shoot apex, leaf sheath, embryonic callus internode, petioles, flower stalks, root or inflorescence, but is not limited thereto.

Introduction and expression of heterologous or chimeric DNA sequences in dicotyledonous (broadleafed) plants such as tobacco, potato and alfalfa has been shown to be possible using the T-DNA of the tumor-inducing (Ti) plasmid of Agrobacterium tumefaciens (See, for example, Umbeck, U.S. Pat. No. 5,004,863, and International application PCT/US93/02480). A construct of the invention may be introduced into a plant cell utilizing A. tumefaciens containing the Ti plasmid. In using an A. tumefaciens culture as a transformation vehicle, it is most advantageous to use a non-oncogenic strain of the Agrobacterium as the vector carrier so that normal non-oncogenic differentiation of the transformed tissues is possible. It is preferred that the Agrobacterium harbors a binary Ti plasmid system. Such a binary system comprises (1) a first Ti plasmid having a virulence region essential for the introduction of transfer DNA (T-DNA) into plants, and (2) a chimeric plasmid. The chimeric plasmid contains at least one border region of the T-DNA region of a wild-type Ti plasmid flanking the nucleic acid to be transferred. Binary Ti plasmid systems have been shown effective to transform plant cells as, for example, described by De Framond (1983, Biotechnology, 1:262) and Hoekema et al. (1983, Nature, 303:179). Such a binary system is preferred inter alia because it does not require integration into the Ti plasmid in Agrobacterium.

Methods involving the use of Agrobacterium include, but are not limited to: (a) co-cultivation of Agrobacterium with cultured isolated protoplasts; (b) transformation of plant cells or tissues with Agrobacterium; or (c) transformation of seeds, apices or meristems with Agrobacterium.

Recently, rice, corn, pineapple and sugarcane, which are monocots, have been shown to be susceptible to transformation by Agrobacterium, for example as described in U.S. Pat. No. 6,037,522, International Publication WO99/36637 and Arencibia et al. (1998, Transgenic Res. 7:213). However, some monocot crop plants have not yet been successfully transformed using Agrobacterium-mediated transformation. The Ti plasmid, however, may be manipulated in the future to act as a vector for these other monocot plants. Additionally, using the Ti plasmid as a model system, it may be possible to artificially construct transformation vectors for these plants. Ti plasmids might also be introduced into monocot plants by artificial methods such as microinjection, or fusion between monocot protoplasts and bacterial spheroplasts containing the T-region, which can then be integrated into the plant nuclear DNA.

In addition, gene transfer can be accomplished by in situ transformation by Agrobacterium, as described by Bechtold et al. (1993, C.R. Acad. Sci. Paris, 316:1194). This approach is based on the vacuum infiltration of a suspension of Agrobacterium cells.

Alternatively, nucleic acids may be introduced using root-inducing (Ri) plasmids of Agrobacterium as vectors.

Cauliflower mosaic virus (CaMV) may also be used as a vector for introducing of exogenous nucleic acids into plant cells (U.S. Pat. No. 4,407,956). CaMV DNA genome is inserted into a parent bacterial plasmid creating a recombinant DNA molecule that can be propagated in bacteria. After cloning, the recombinant plasmid again may be cloned and further modified by introduction of the desired nucleic acid sequence. The modified viral portion of the recombinant plasmid is then excised from the parent bacterial plasmid, and used to inoculate the plant cells or plants.

Nucleic acids can also be introduced into plant cells by electroporation as, for example, described by Fromm et al. (1985, Proc. Natl. Acad. Sci., U.S.A, 82:5824) and Shimamoto et al. (1989, Nature 338:274-276). In this technique, plant protoplasts are electroporated in the presence of vectors or nucleic acids containing the relevant nucleic acid sequences. Electrical impulses of high field strength reversibly permeabilise membranes allowing the introduction of nucleic acids. Electroporated plant protoplasts reform the cell wall, divide and form a plant callus.

Another method for introducing nucleic acids into a plant cell is high velocity ballistic penetration by small particles (also known as particle bombardment or microprojectile bombardment) with the nucleic acid to be introduced contained either within the matrix of small beads or particles, or on the surface thereof as, for example described by Klein et al. (1987, Nature 327:70). Although typically only a single introduction of a new nucleic acid sequence is required, this method particularly provides for multiple introductions.

Alternatively, nucleic acids can be introduced into a plant cell by contacting the plant cell using mechanical or chemical means. For example, a nucleic acid can be mechanically transferred by microinjection directly into plant cells by use of micropipettes. Alternatively, a nucleic acid may be transferred into the plant cell by using polyethylene glycol which forms a precipitation complex with genetic material that is taken up by the cell.

Also contemplated are silicon carbide or tungsten whiskers, for example as described in U.S. Pat. No. 5,302,523.

There are a variety of methods known currently for transformation of monocotyledonous plants. Presently, preferred methods for transformation of monocots are microprojectile bombardment of explants or suspension cells, and direct DNA uptake or electroporation as, for example, described by Shimamoto et al. (1989, supra). Transgenic maize plants have been obtained by introducing the Streptomyces hygroscopicus bar gene into embryogenic cells of a maize suspension culture by microprojectile bombardment (Gordon-Kamm, 1990, Plant Cell, 2:603-618). The introduction of genetic material into aleurone protoplasts of other monocotyledonous crops such as wheat and barley has been reported (Lee, 1989, Plant Mol. Biol. 13:21-30). Wheat plants have been regenerated from embryogenic suspension culture by selecting only the aged compact and nodular embryogenic callus tissues for the establishment of the embryogenic suspension cultures (Vasil, 1990, Bio/Technol. 8:429-434). The combination with transformation systems for these crops enables the application of the present invention to monocots. These methods may also be applied for the transformation and regeneration of dicots. Transgenic sugarcane plants have been regenerated from embryogenic callus as, for example, described by Bower et al. (1996, Molecular Breeding 2:239-249).

Alternatively, a combination of different techniques may be employed to enhance the efficiency of the transformation process, e.g., bombardment with Agrobacterium coated microparticles (EP-A-486234) or microprojectile bombardment to induce wounding followed by co-cultivation with Agrobacterium (EP-A-486233).

### Plant Regeneration

The methods used to regenerate transformed cells into differentiated plants are not critical to this invention, and any method suitable for a target plant can be employed. Normally, a plant cell is regenerated to obtain a whole plant following a transformation process.

The term “regeneration” as used herein means growing a whole, differentiated plant from a plant cell, a group of plant cells, a plant part (including seeds), or a plant piece (e.g., from a protoplast, callus, or tissue part).

Regeneration from protoplasts varies from species to species of plants, but generally a suspension of protoplasts is first made. In certain species, embryo formation can then be induced from the protoplast suspension, to the stage of ripening and germination as natural embryos. The culture media will generally contain various amino acids and hormones, necessary for growth and regeneration. Examples of hormones utilized include auxins and cytokinins. It is sometimes advantageous to add glutamic acid and proline to the medium, especially for such species as corn and alfalfa. Efficient regeneration will depend on the medium, on the genotype, and on the history of the culture. If these variables are controlled, regeneration is reproducible. Regeneration also occurs from plant callus, explants, organs or parts. Transformation can be performed in the context of organ or plant part regeneration as, for example, described in Methods in Enzymology, Vol. 118 and Klee et al. (1987, Annual Review of Plant Physiology, 38:467), which are incorporated herein by reference. Utilizing the leaf disk-transformation-regeneration method of Horsch et al. (1985, Science, 227:1229, incorporated herein by reference), disks are cultured on selective media, followed by shoot formation in about 2-4 weeks. Shoots that develop are excised from calli and transplanted to appropriate root-inducing selective medium. Rooted plantlets are transplanted to soil as soon as possible after roots appear. The plantlets can be repotted as required, until reaching maturity.

In vegetatively propagated crops, the mature transgenic plants are propagated by the taking of cuttings or by tissue culture techniques to produce multiple identical plants. Selection of desirable transgenotes is made and new varieties are obtained and propagated vegetatively for commercial use.

In seed propagated crops, the mature transgenic plants can be self-crossed to produce a homozygous inbred plant. The inbred plant produces seed containing the newly introduced heterologous gene(s). These seeds can be grown to produce plants that would produce the selected phenotype, e.g., early flowering.

Parts obtained from the regenerated plant, such as flowers, seeds, leaves, branches, fruit, and the like are included in the invention, provided that these parts comprise cells that have been transformed as described. Progeny and variants, and mutants of the regenerated plants are also included within the scope of the invention, provided that these parts comprise the introduced nucleic acid sequences.

It will be appreciated that the literature describes numerous techniques for regenerating specific plant types and more are continually becoming known. Those of ordinary skill in the art can refer to the literature for details and select suitable techniques without undue experimentation.

### Characterization

To confirm the presence of the heterologous nucleic acid in the regenerating plants, a variety of assays may be performed. Such assays include, for example, “molecular biological” assays well known to those of skill in the art, such as Southern and Northern blotting and PCR; a protein expressed by the heterologous DNA may be analysed by western blotting, high performance liquid chromatography or ELISA (e.g., nptII) as is well known in the art.

Examples of various methods applicable to characterization of transgenic plants are provided in Chapters 9 and 11 of PLANT MOLECULAR BIOLOGY A Laboratory Manual Ed. M.S. Clark (Springer-Verlag, Heidelberg, 1997), which chapters are herein incorporated by reference

So that the invention may be readily understood and put into practical effect, the following non-limiting Examples are provided.

### EXAMPLES

### Example 1

Materials and Methods

LongSAGE Analysis

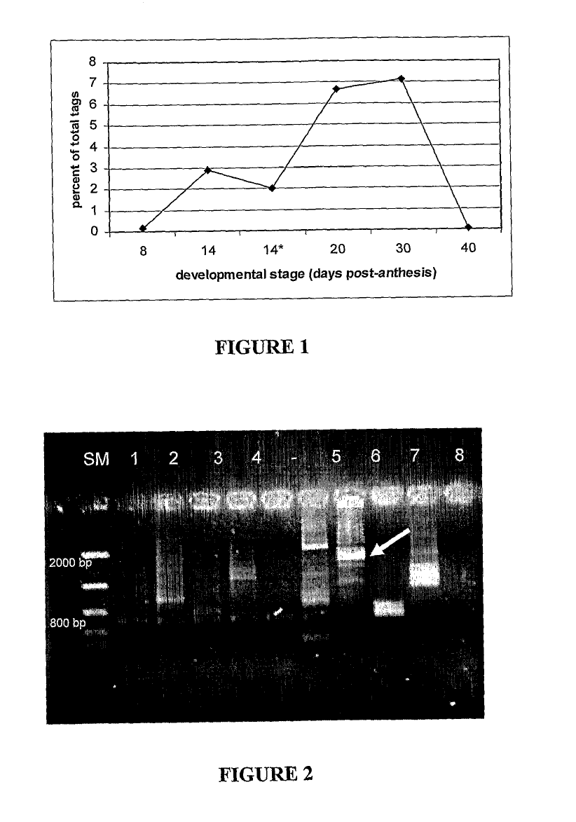

LongSAGE libraries were constructed from pooled wheat (Triticum aestivum cv. Banks) seed samples for each of the developmental stages 8, 14, 20 and 30 day post anthesis (dpa) and also for mature seed according to McIntosh et al. (2006). The present study sought to define the most abundant LongSAGE transcripts (or tags) present in both the 20 and 30 dpa libraries, and to then determine a suitable candidate for further gene promoter studies. Approximately fifty LongSAGE tags were annotated (data not shown) through blastn comparisons with Genbank plant EST sequences via the NCBI website (www.ncbi.nlm.nih.gov). All tags matching to glutenin or gliadin genes, which are already well-characterised and in most cases patented, were not considered further as promoter candidates. A single LongSAGE tag with the sequence CATGT TGTTC CGTGT AGTAC C (referred to herein as TagA) was selected for further analyses due to its high expression levels. TagA was the second most abundant tag within the 30 dpa library, and was also highly represented in several other libraries (FIG. 1).

### EST Library Construction

LongSAGE tags were compared manually to full length EST sequences generated in our laboratory, derived from the same wheat seed (14 dpa) sample. This EST library was constructed with the BD Biosciences Clontech (Foster City, Calif.) Creator™ SMART™ cDNA library construction kit using the pDNR-LIB vector. Approximately one thousand EST clones were sequenced, with those matching TagA incorporated into further analyses.

### Genome Walking

Upstream promoter sequences for this transcript were obtained from wheat (cv. Bob White) genomic DNA using the BD GenomeWalker™ Universal Kit (BD Biosciences Clontech) as per instructions. Reverse gene specific primers for PCR amplification were designed from the 5′ end of EST sequences matching TagA using Clone Manager Professional Suite v8. Prom1_{—}1a (GGCTA GCACC ATGAT GGTAG CAACA C) was used for the first round of PCR reactions, for the second round of PCR reactions Prom1_{—}1b (TGGCT GCCTA TCTCG TCACA CTCTT C) was used. Approximately 5 μl of each reaction was electrophoresed on an agarose gel and the corresponding PCR products visualised via UV transillumination techniques using ethidium bromide. For comparison, 5 μl of Invitrogen (Carlsbad, Calif.) E-gel® Low Range Quantitative DNA Ladder was also run (FIG. 2).

### Gel-Purification and Cloning

Approximately 20 μlof each second round PCR product was mixed with 5 μl of 6×TAE loading dye and electrophoresed on a 1.5% NuSieve® GTG® Agarose (Cambrex Bio Science, Rockland Me.) low melt gel in 1×TAE buffer with ethidium bromide at 60v for around 2 h. DNA bands present in the gel were visualised with a hand-held UVP (Upland, Calif.) UVM-57 302 nm transilluminator and the brightest band from each lane excised with a clean scalpel blade. Gel slices were melted at 72° C. for 1 min, and then held at 37° C. until required for vector ligation. Half volume ligations were performed with Promega (Madison, Wis.) pGEM®-T Easy Vector as per instructions, with ligations placed on ice to cool for 5 min (following the addition of the molten gel PCR products) and then incubated overnight at room temperature. Ligations were cloned into One Shot® TOP10 Electrocomp™ E. coli cells (Invitrogen) according to the manufacturers instructions, with ligations pre-heated to 72° C. for 1 min and then held at 37° C. Transformations were spread onto LB Amp/IPTG/X-gal agar plates and incubated overnight at 37° C. Eight positive colonies from each transformation were amplified using half reactions of the TempliPhi™ DNA Template Amplification Kit from Amersham Biosciences (Buckinghamshire, UK) and sequenced using M13 forward and reverse primers. The resulting data confirmed that the DNA band indicated by an arrow in FIG. 2 corresponded to the EST sequence data from which the PCR primers were derived (data not shown). Approximately 24 colonies derived from this transformation were subsequently picked and cultured individually in LB with Amp overnight at 37° C. Plasmid purification was performed using the Qiagen (Hilden, Germany) QIAprep spin Miniprep Kit as per instructions, with DNA concentration analysed using a Nanodrop ND-1000 spectrophotometer (NanoDrop Technologies, Wilmington Del.).

### Sequencing and Data Analyses

Sequencing was performed using an Applied Biosystems (Foster City, Calif.) BigDye® Terminator v3.1 Cycle Sequencing Kit. The M13 forward primer was used to obtain all EST sequences, whereas both the M13 forward and reverse primers were required for all colony and miniprep sequencing. Automated DNA sequencing was carried out with an ABI 3730×148 capillary DNA analyser (Applied Biosystems). Data from both forward and reverse sequences were manually checked and combined to form a single sequence per colony or miniprep. Miniprep sequences were then aligned using ClustalW (Higgins et al. 1994) in MEGA v3.1 (Kumar et al. 2004) and checked for homology with the original ESTs. A single consensus sequence was assembled from all matching miniprep promoter sequences, and screened for regulatory elements via the PLACE (http://www.dna.affrc.go.jp/PLACE/) (Prestridge 1991; Higo et al. 1999) and PlantCARE (http://bioinformatics.psb.ugent.be/) (Lescot et al. 2002) servers. Hypotheses regarding gene function were made by analysing the putative coding region of EST sequences via the web-based PredictProtein server (http://www.predictprotein.org/) (Rost et al. 2004).

### Results

### EST Analyses

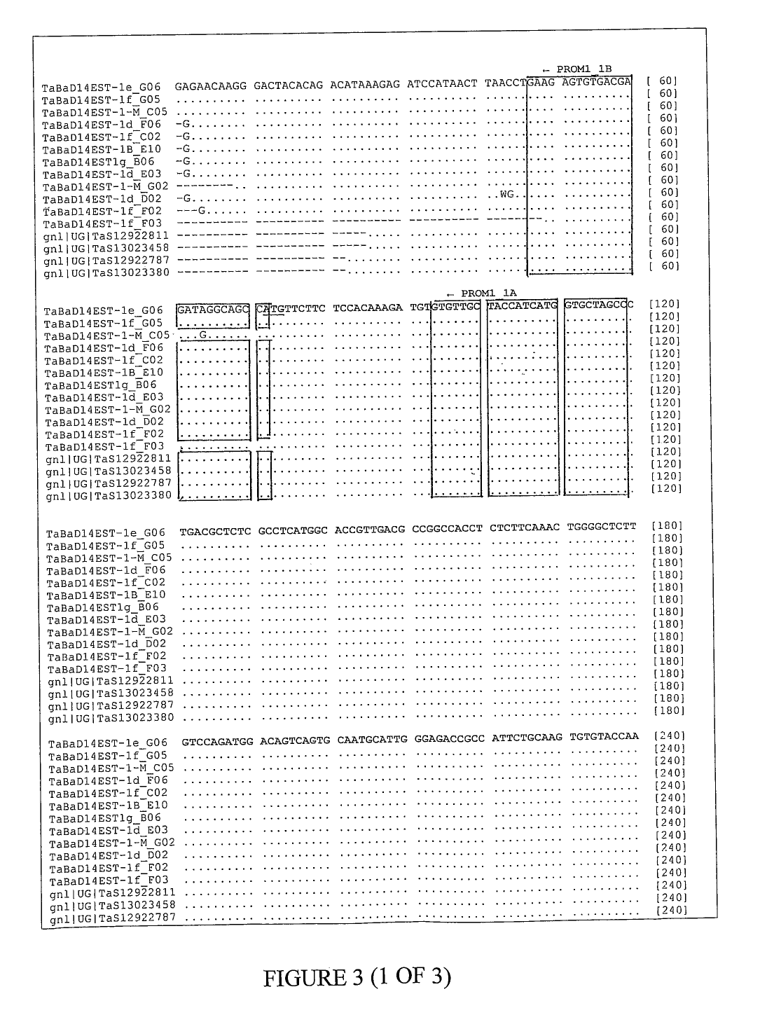

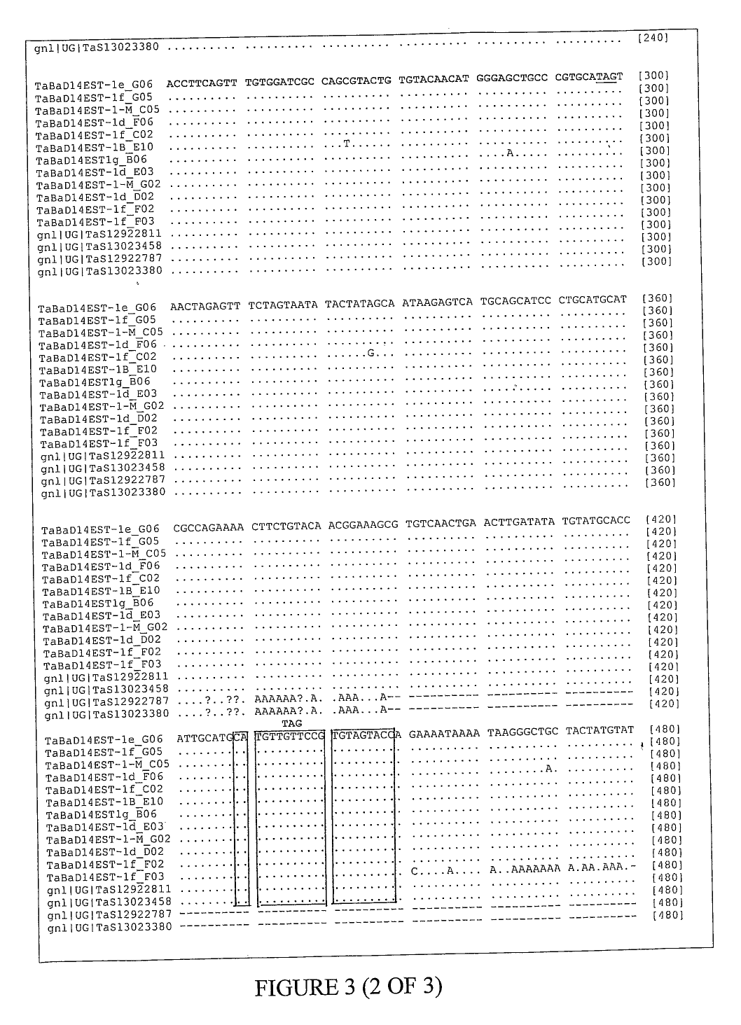

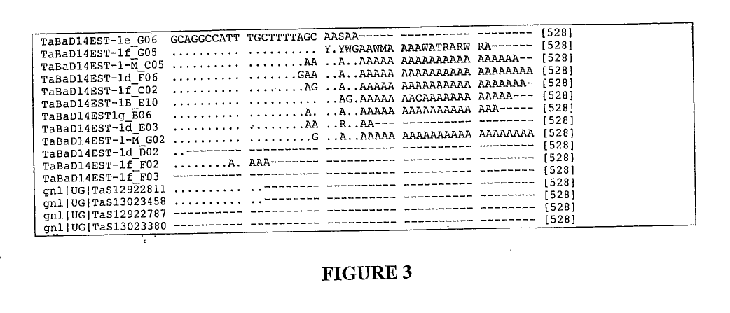

Web-based comparisons found TagA matched EST sequences corresponding to Unigene cluster Ta.2025 (four sequences) which were downloaded and aligned with similar sequences present in our wheat EST library (FIG. 3). Very little divergence was observed among these sequences, and observed differences in transcript length in relation to the terminal polyA sequence do not alter the putative translation. Due to the short and unambiguous nature of these EST transcripts, assigning putative transcription and translation initiation sites was relatively straightforward. In this regard, mRNA transcripts from higher plants tend to have AU-rich leader sequences (<200 bp in length) and translation usually begins at the first AUG codon (Joshi et al. 1997).

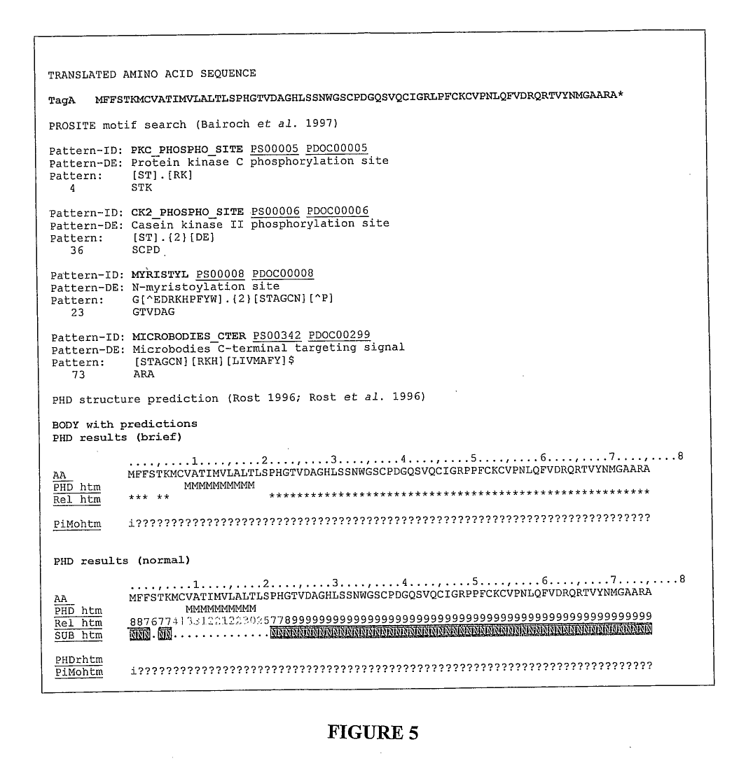

A putative function for the corresponding gene was sought through various nucleotide and translated amino acid blast comparisons of EST sequences with DNA databases available on the web (eg. NCBI, PlantGDB, TIGR) however no significant homology to any gene of known function was observed. Motif and structural prediction of the putative translated amino acid sequence via PredictProtein however, indicates this gene probably encodes a small microbody-associated protein (FIG. 5).

### Promoter Sequence Analysis

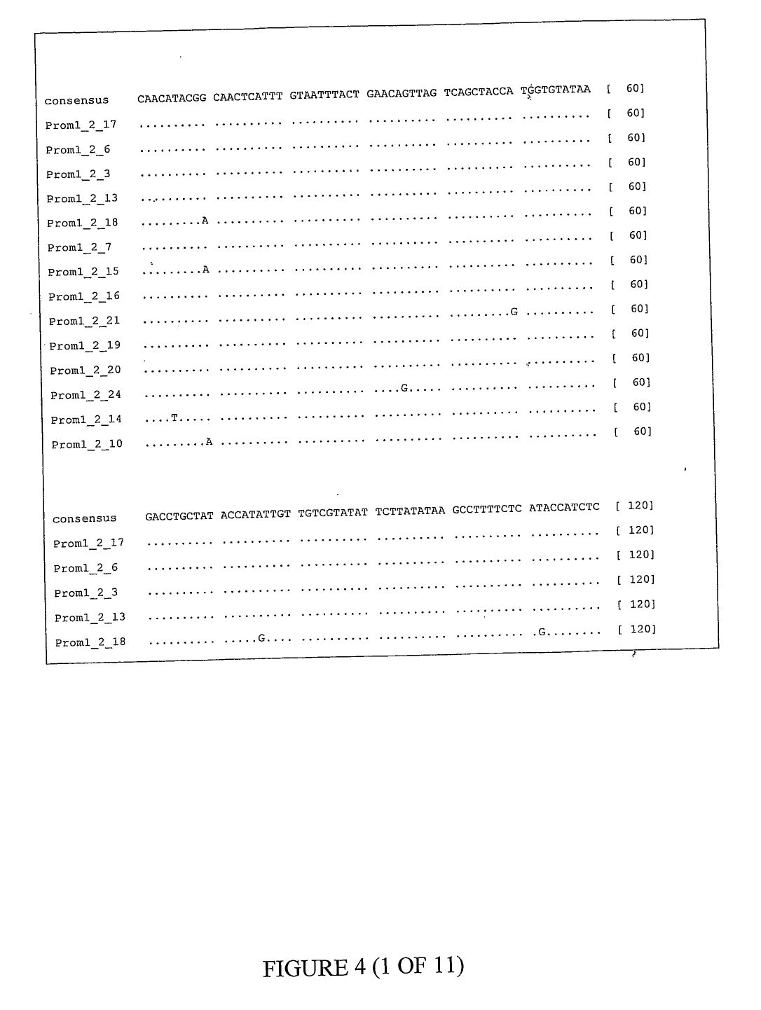

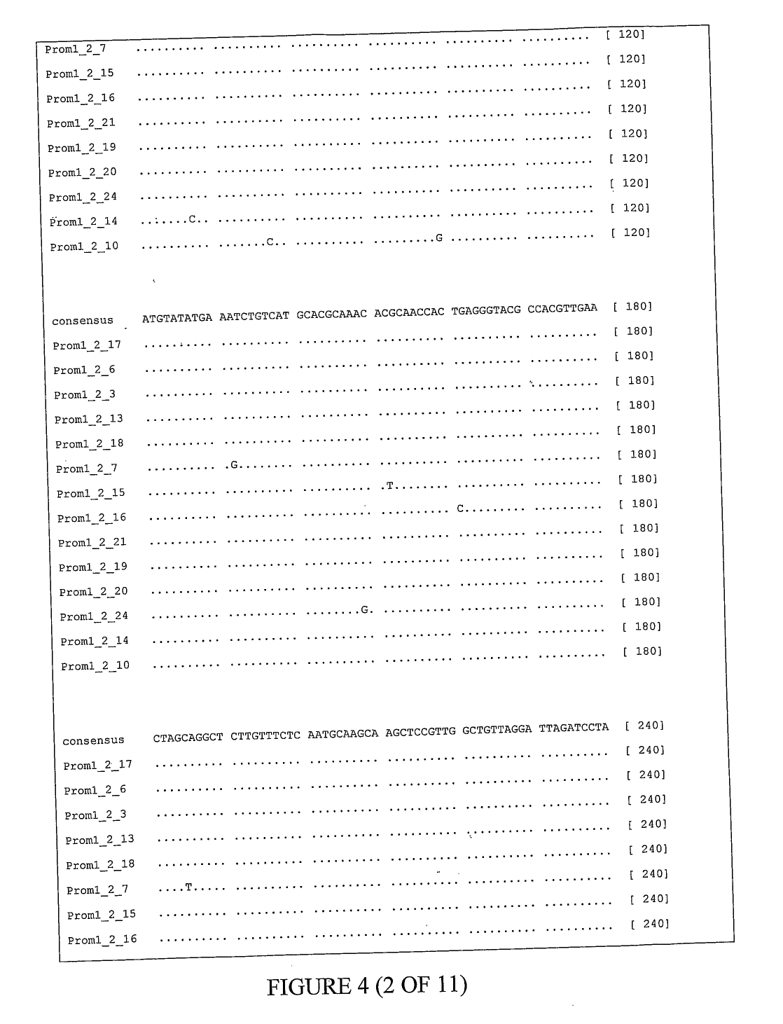

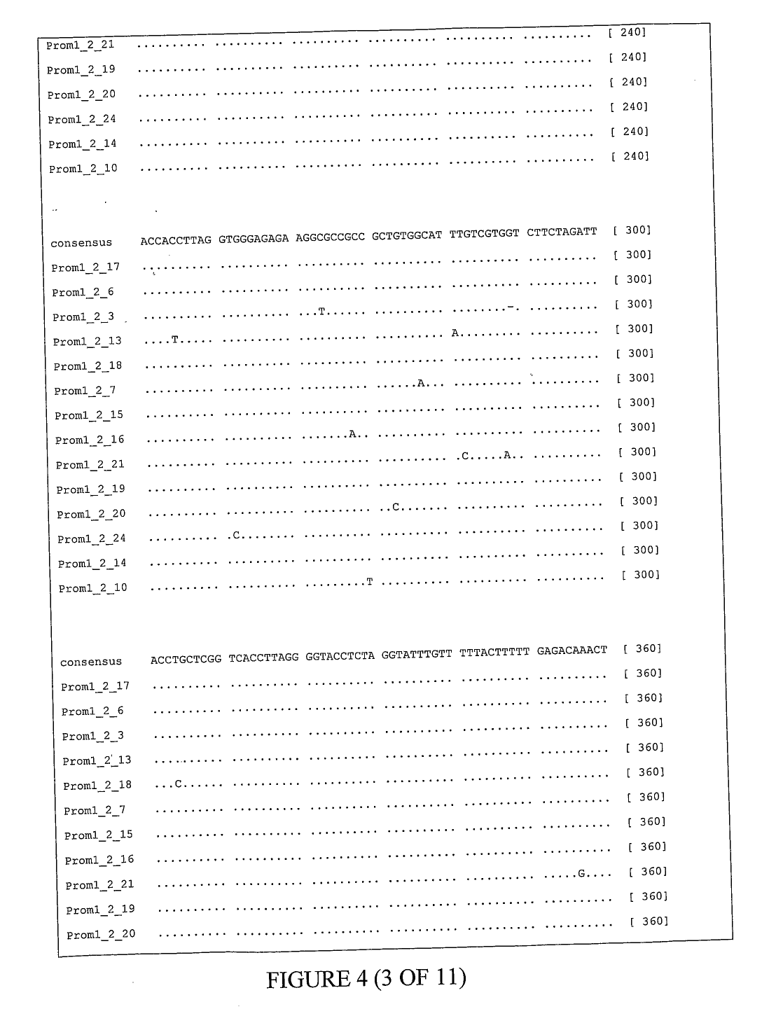

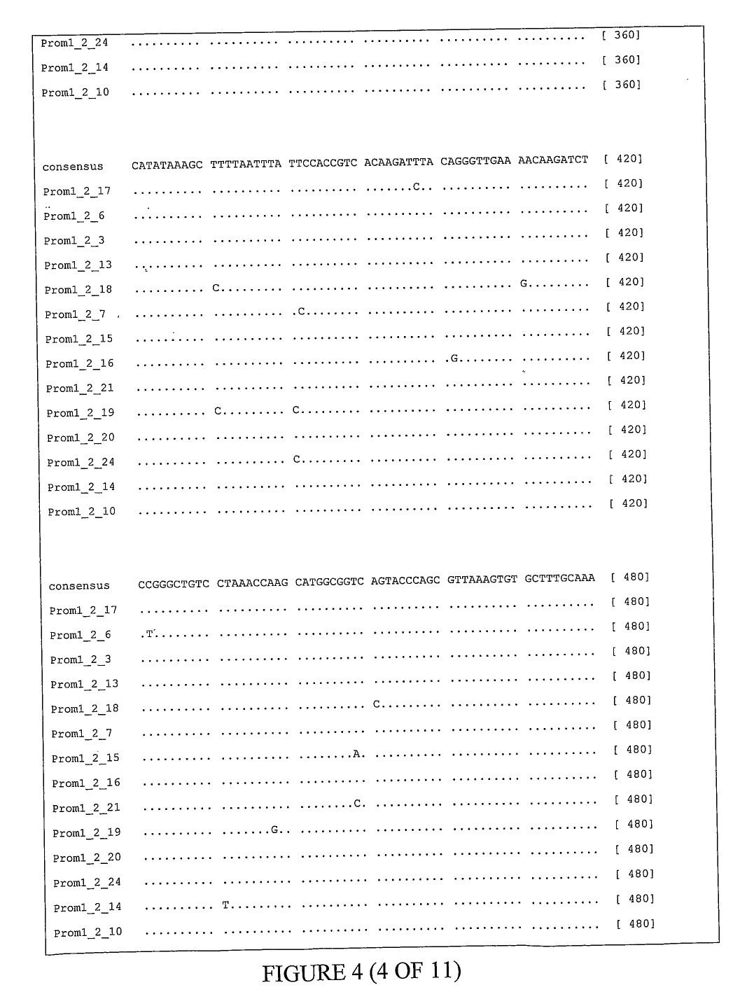

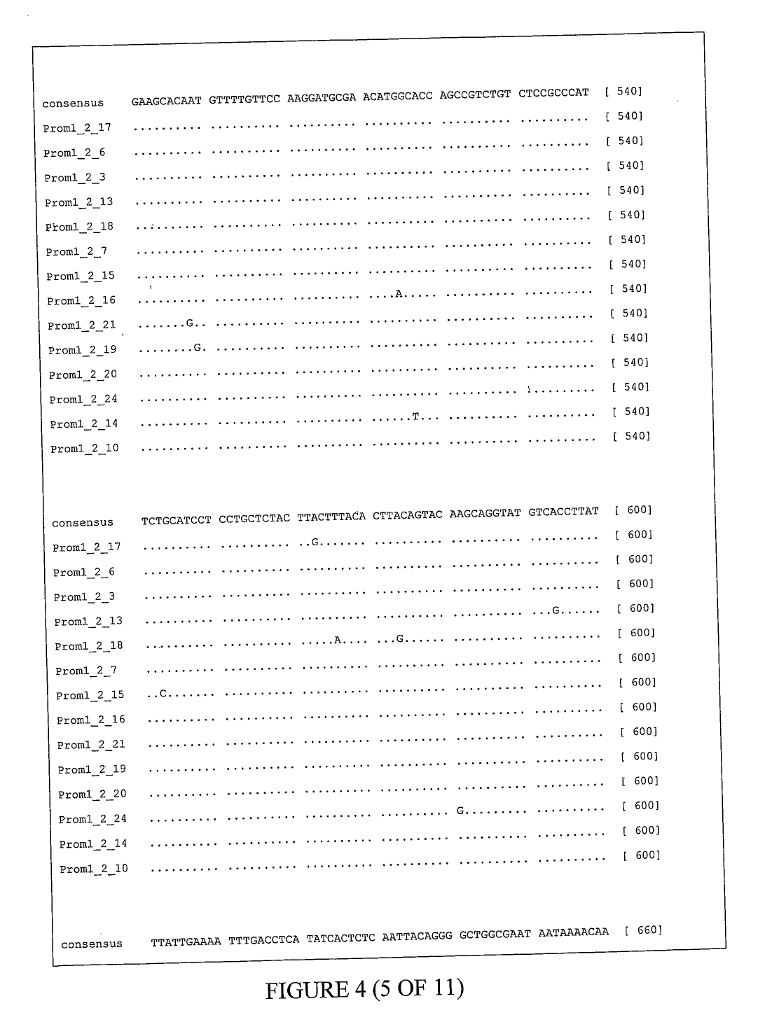

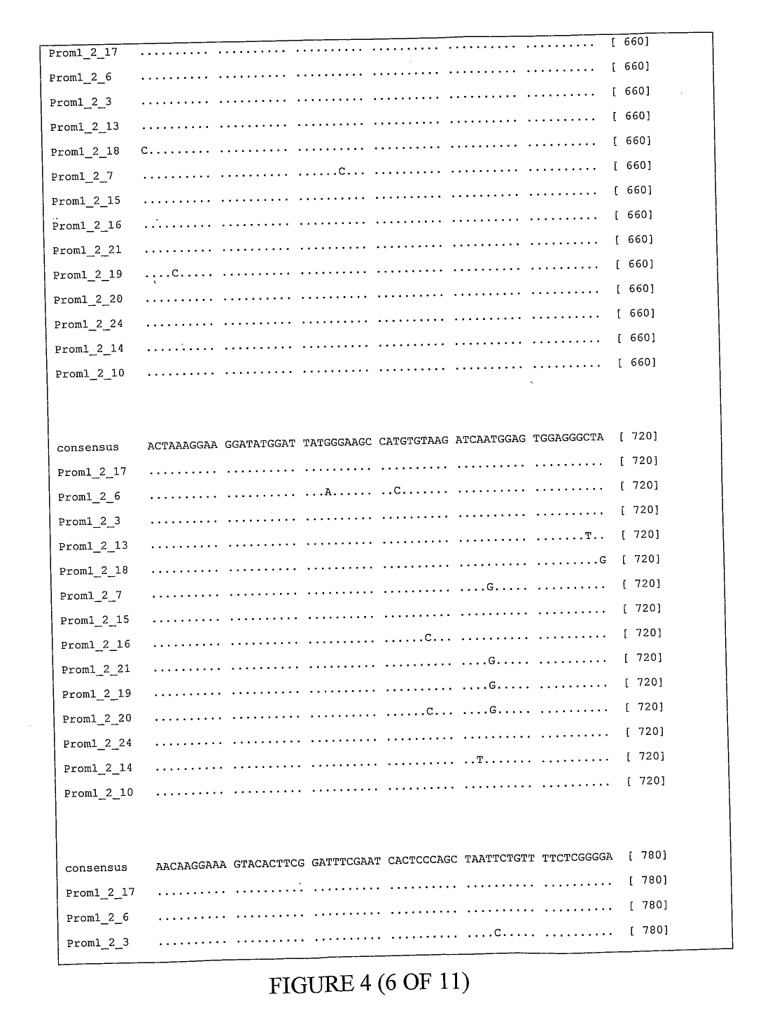

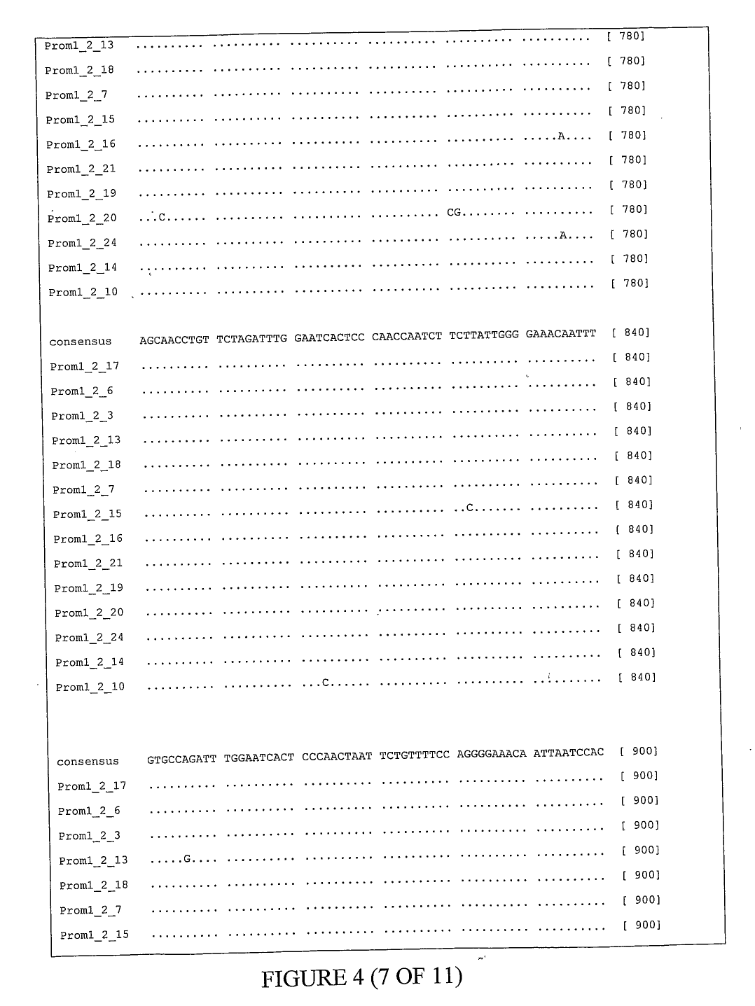

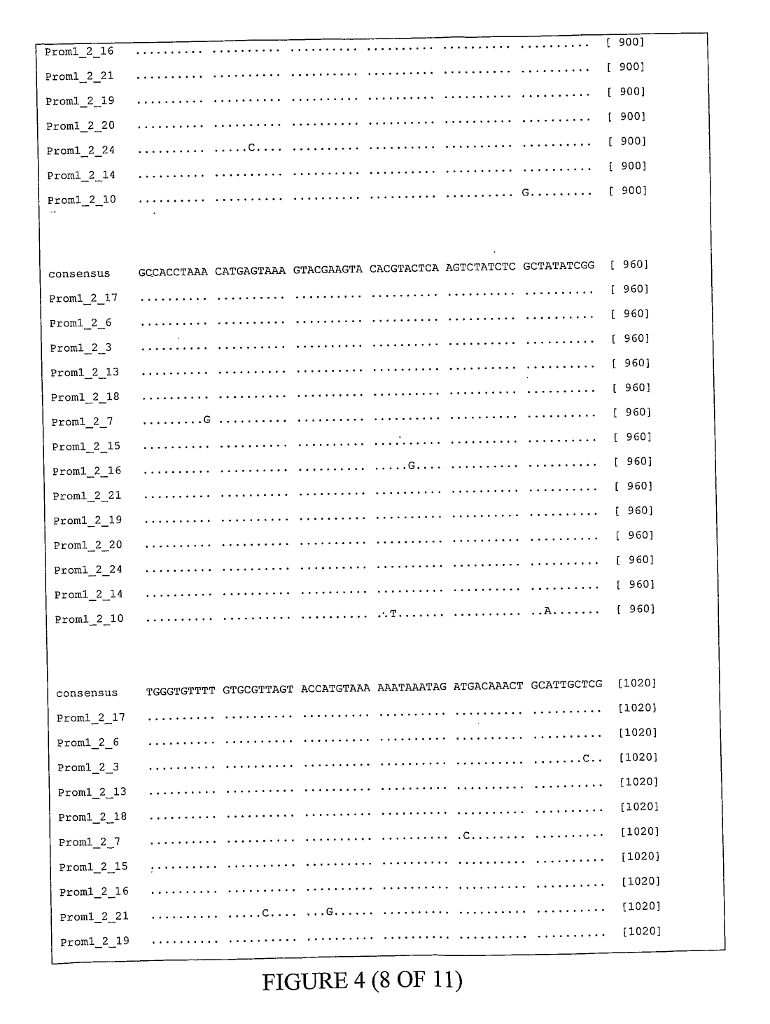

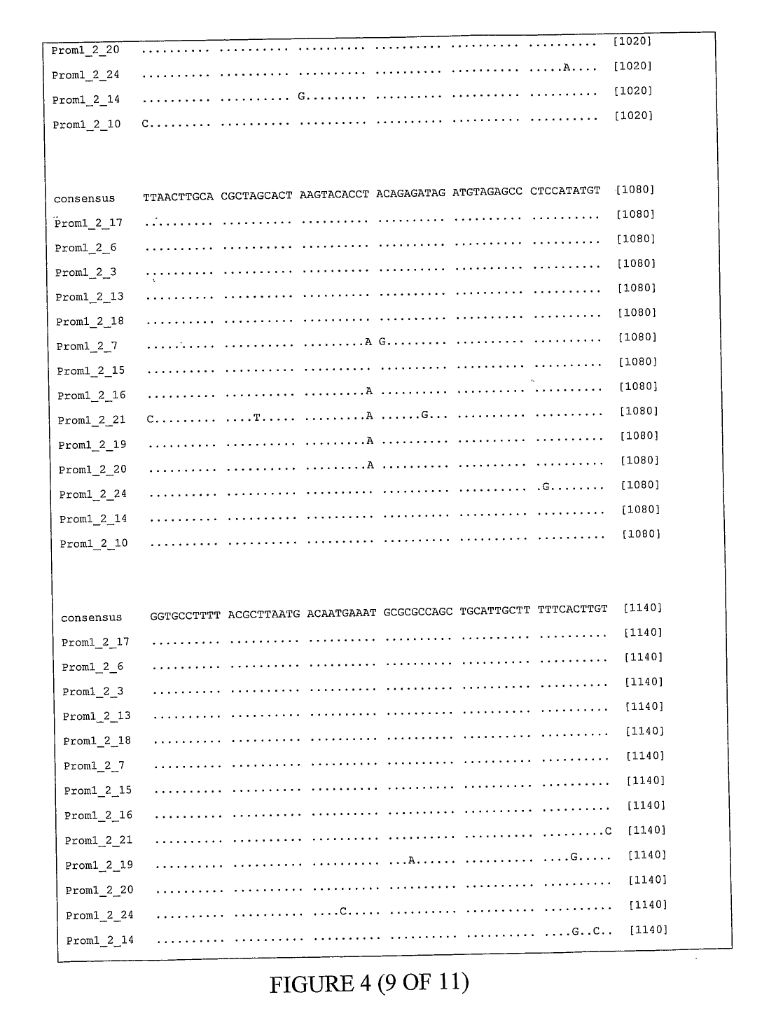

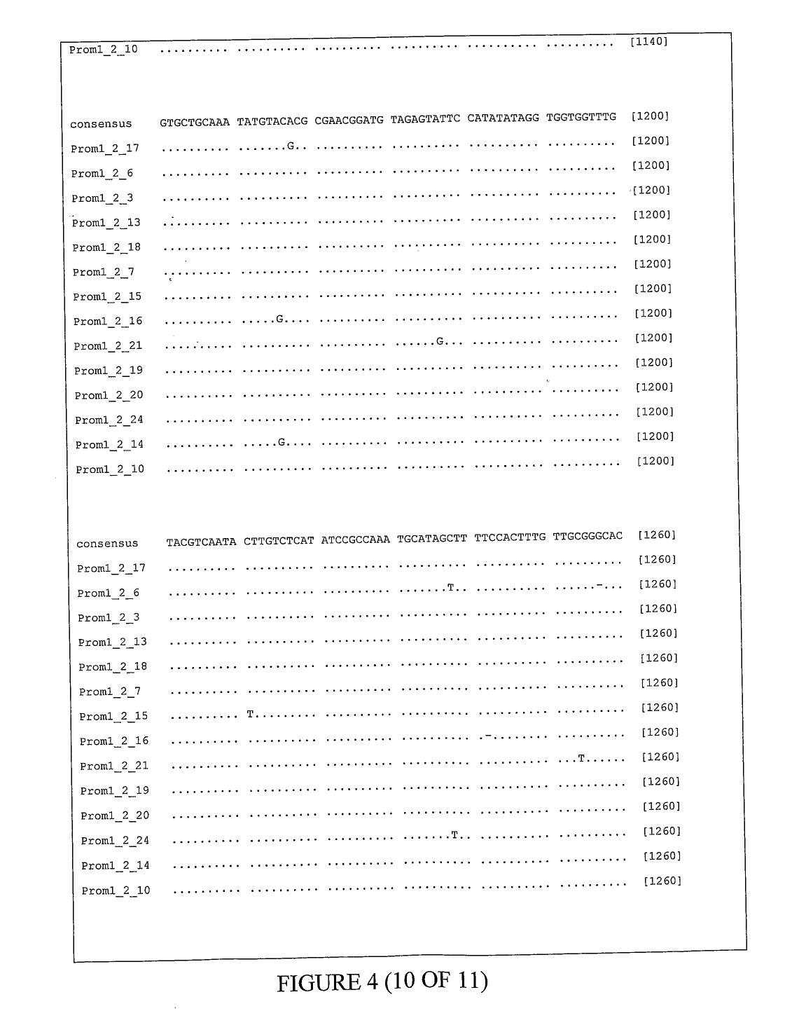

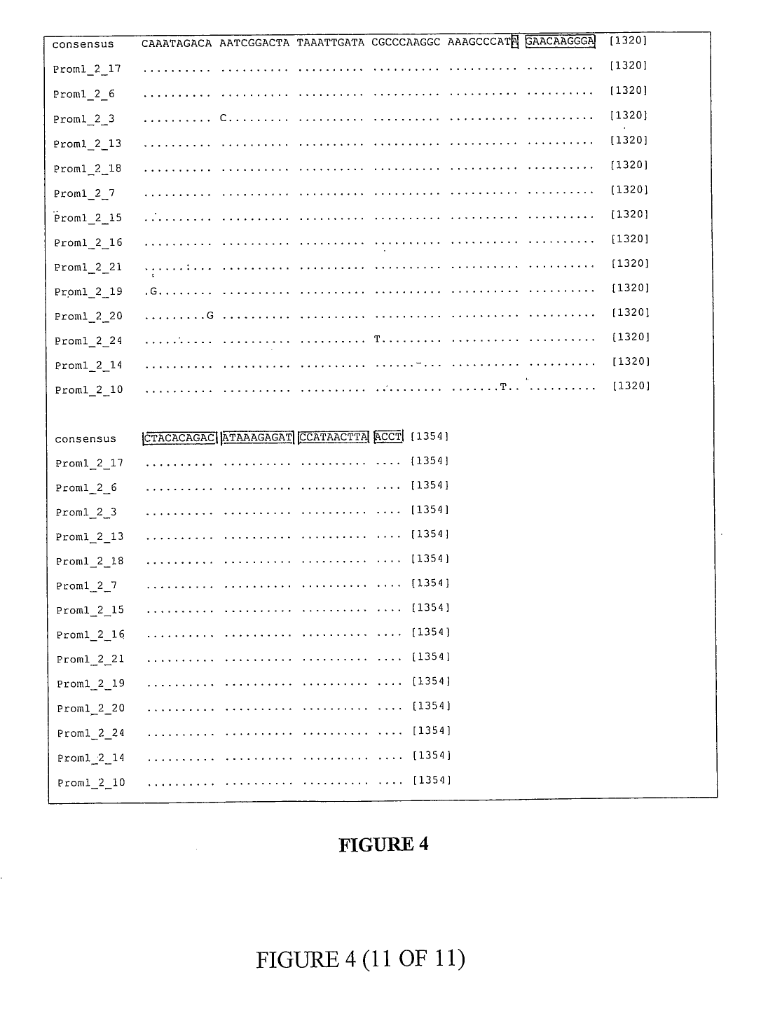

A genomic DNA fragment for a putative TagA promoter sequence of around 1300 bp was successfully cloned and sequenced. A number of highly similar sequences (no greater than 2.1% divergence) were obtained that were identical to the 5′ end of the corresponding EST sequences. These sequences were aligned and a consensus sequence determined (FIG. 4). Analysis of the consensus sequence for regulatory elements revealed the presence of likely TATA and CAAT boxes, and several possible endosperm elements (Table 1). Also present were several elements involved in early dehydration and light response, which suggests the corresponding protein may be involved in osmoprotection. Additional gene regulatory elements that were detected using the PLACE server are listed in Table 2.

### Discussion

In this study we have determined a novel promoter sequence for an endogenous wheat gene that is highly expressed in the developing seed, which represents the first step in developing a new expression cassette for successful genetic transformation of wheat and other cereal grains. Interestingly, regulatory elements associated with endosperm gene expression were detected within the promoter sequence. It is therefore anticipated that with further research this promoter sequence may be used to deliver high expression of heterologous genes in the endosperm of seeds from wheat and other plant species. Similar studies with this goal in mind have targeted the promoter of a high molecular weight (HMW) glutenin subunit gene, which was found to have endosperm-specific expression (Lamacchia et al. 2001).

Analyses of the corresponding gene transcript further suggest the translated protein may be secreted to protein microbodies within the endosperm. In this regard, protein signal peptides encoded by the transcribed sequence described in this study (the ARA microbody signal sequence particularly) may be used to further direct recombinant proteins down the secretory pathway, and into protein bodies within the endosperm. N-terminal and C-terminal signal sequences have been used to direct localisation of recombinant proteins in the wheat endosperm, most notably human serum albumen (Arcalis et al. 2004). Further studies are required to determine localisation of the protein described in this study and its possible function.

### Example 2

### Construction of Genetically-Modified Wheat by Agrobacterium-Mediated Transformation

### Constructs

The construct pEvec202Nnos will be used to prepare all the constructs used for transformation of wheat. Transcriptional fusions between the promoter sequences of the present invention, the CaMV35S promoter and the gfps65T henceforth referred to as gfp sequence respectively, will be generated by PCR, such as in described in Furtado, A. and Henry, R. J. (2006), Plant Biotechnology Journal 3: 421-434.

### Agrobacterium-Mediated Transformation of Barley and Rice

Transgenic wheat will be generated by Agrobacterium-mediated transformation of embryogenic callus. The embryo will be isolated from wheat seed under sterile conditions. Agrobacterium tumefaciens transformed with constructs will be grown overnight in MGL medium. For inoculation, an Eppendorf pipette will be used to place drops of the Agrobacterium culture on the cut side of the immature embryos. After incubation of the plates for about two days in the dark at 24° C., the embryos will be transferred into plates containing BCI-DM medium supplemented with hygromycin and timentin. After about six weeks of dark incubation, with transfers in fresh medium every two weeks, the embryogenic callus produced will be transferred to FHG medium supplemented with hygromycin. Regenerated shoots will be transferred into BCI medium for development of roots before transfer in soil. Detection of green fluorescence from GFP will be carried out using a compound microscope equipped with an attachment for fluorescence observations.

To determine presence of transgene, PCR screening of transgenic plants will be carried out using purified genomic DNA. All hygromycin-resistant plants will be screened for the gfp-nos sequence by PCR (such as according to Furtado, A. and Henry, R. J. (2006), Plant Biotechnology Journal 3: 421-434). Southern-blot hybridisation will be carried out essentially according to established procedures (Maniatis et al., 1982). Genomic DNA from non-transformed or transformed plants will be digested with Hind III and checked for digestion before resolving on an agarose gel, followed by transfer onto a nylon membrane (Nylon-hybond, Roche, Germany). Hybridisation will be carried out using Dig-labelled probe corresponding to the gfp gene, followed by signal development using the Dig-detection system (Roche, Germany).

### Example 3

### Construction of Genetically-Modified Wheat by Particle Bombardment

### Constructs Used for Particle Bombardment

The plasmid pAGN, a pGEM3Zf+ based vector (Promega Corporation, MI, USA) and containing a synthetic variant of the green fluorescent protein gene (gfpS65T) (Patterson et al., 1997) and nos terminator sequence, will be used as the cloning vector to generate the promoter construct. The promoter.gfp.nos construct will be prepared as a transcriptional fusion of the promoter with the gfpS65T henceforth referred as the gfp gene. The plasmid pAGN will also be used as the cloning vector to generate the gene constructs pUbi.gfp.nos, pCaMV35S.gfp.nos which contain the maize ubiquitin, the cauliflower mosaic virus 35S RNA promoter, linked to the gfp gene and nos terminator sequence. Plasmid pDP687 will be used as a control to check for successful particle-bombardment and viability of cells, and contains the cauliflower mosaic virus 35S RNA promoter (CaMV35S) which controls the constitutive expression of two genes, each encoding transcription factors which regulate synthesis of the red anthocyanin pigment.

Tissue preparation, particle bombardment and incubation conditions will be performed such as described in Furtado, A. and Henry, R. J. (2006), Plant Biotechnology Journal 3: 421-434.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, patent and scientific literature referred to herein is incorporated herein by reference.

### REFERENCES

Arcalis, E., S. Marcel, F. Altmann, D. Kolarich, G. Drakakaki, R. Fischer, P. Christou and E. Stoger (2004). Unexpected Deposition Patterns of Recombinant Proteins in Post-Endoplasmic Reticulum Compartments of Wheat Endosperm 10.1104/pp. 104.050153. Plant Physiol. 136(3): 3457-3466.
*Bairoch, A., P. Bucher and K. Hofman (1997). PROSITE. Nucleic Acids Research 25: 217-221.
Brinch-Pedersen, H., F. Hatzack, L. D. Sorensen and P. B. Holm (2003). Concerted action of endogenous and heterologous phytase on phytic acid degradation in seed of transgenic wheat (Triticum aestivum L.). Transgenic Res. 12: 649-659.
Drea, S., D. J. Leader, B. C. Arnold, P. Shaw, L. Dolan and J. H. Doonan (2005). Systematic Spatial Analysis of Gene Expression during Wheat Caryopsis Development. Plant Cell 17(8): 2172-2185.
Higgins, D., J. Thompson, T. Gibson, J. D. Thompson, D. G. Higgins and G. T. J. (1994). CLUSTAL W: improving the sensitivity of progressive multiple sequence alignment through sequence weighting, position-specific gap penalties and weight matrix choice. Nucleic Acids Research 22: 4673-4680.
Higo, K., Y. Ugawa, M. Iwamoto and T. Korenaga (1999). Plant cis-acting regulatory DNA elements (PLACE) database:1999. Nucleic Acids Research 27(1): 297-300.
Joshi, C. P., H. Zhou, X. Huang and V. L. Chiang (1997). context sequences of translation initiation codon in plants. Plant Molecular Biology 35: 993-1001.
Kumar, S., K. Tamura and M. Nei (2004). MEGA3: Integrated software for Molecular Evolutionary Genetics Analysis and sequence alignment. Briefings in Bioinformatics 5: 150-163.
Lamacchia, C., P. R. Shewry, N. Di Fonzo, J. L. Forsyth, N. Harris, P. A. Lazzeri, J. A. Napier, N. G. Halford and P. Barcelo (2001). Endosperm-specific activity of a storage protein gene promoter in transgenic wheat seed. Journal of Experimental Botany 52(355): 243-250.
Lescot, M., P. Déhais, G. Thijs, K. Marchal, Y. Moreau, Y. Van de Peer, P. Rouzé and S. Rombauts (2002). PlantCARE, a database of plant cis-acting regulatory elements and a portal to tools for in silico analysis of promoter sequences. Nucleic Acids Research 30(1): 325-327.
McIntosh, S., L. Watson, P. Bundock, A. C. Crawford, J. White, G. Cordeiro, D. Barbary, L. Rooke and R. J. Henry (2006). SAGE of the most abundant transcripts in the developing wheat caryopsis. The Plant Journal in press.
Prestridge, D. S. (1991). SIGNAL SCAN: A computer program that scans DNA sequences for eukaryotic transcriptional elements. CABIOS 7: 203-206.
*Rost, B. (1996). PHD. Methods in Enzymology 266: 525-539.
*Rost, B., P. Fariselli and R. Casadio (1996). PHDhtm. Protein Science 7: 1704-1718.
Rost, B., G. Yachdev and J. Liu (2004). The PredictProtein Server. Nucleic Acids Research 32 (Web Server issue): W321-W326.
Shewry, P. R. and H. D. Jones (2005). Transgenic wheat: where do we stand after the first 12 years? Annals of Applied Biology 147(1): 1-14.
Stöger, E., J. K. C. Ma, R. Fischer and P. Christou (2005). Sowing the seeds of success: pharmaceutical proteins from plants. Current Opinion in Biotechnology 16(2): 167-173.
Stöger, E., M. Parker, P. Christou and R. Casey (2001). Pea Legumin Overexpressed in Wheat Endosperm Assembles into an Ordered Paracrystalline Matrix. Plant Physiol. 125: 1732-1742.

### TABLES

TABLE 1
Possible plant regulatory elements found within the promoter
consensus sequence.

| Motif | Strand | Distance* | Sequence |
|---|---|---|---|
| Plastid element | + | −2 | ATAGAA |
| TATA box | + | −31 | TATAAAT |
| CAAT box | + | −49 | CAAAT |
| Light responsive element | + | −65 | ACTTTG |
| E box | + | −83 | CAAATG |
| E box | + | −176 | CACTTG |
| E box | + | −193 | CAGCTG |
| E box | + | −236 | CATATG |
| Circadian element | + | −371 | CAANNNNATC |
| E box | + | −619 | CATGTG |
| Endosperm specific element | + | −654 | AACAAAC |
| Prolamine box | + | −835 | TGCAAAGA |
| E box | + | −1033 | CATTTG |
| Unfolded protein response | + | −1153 | CCNNNNNNNNNNNNCCACG |
| Dehydration-responsive element | + | −1072 | CTAACCA |
| Storage protein element | + | −1164 | CAAACAC |

*bp from transcription start

TABLE 2
Additional gene regulatory elements detected in the consensus
promoter sequence

| | | |
|---|---|---|
| Factor or Site Name | Loc.(Str.) | Signal Sequence |

| | | | | |
|---|---|---|---|---|
| −10PEHVPSBD | site | 87 | (+) | TATTCT |
| −300CORE | site | 563 | (−) | TGTAAAG |
| −300ELEMENT | site | 562 | (−) | TGHAAARK |
| −300ELEMENT | site | 1128 | (−) | TGHAAARK |
| 2SSEEDPROTBANAPA | site | 145 | (+) | CAAACAC |
| AACACOREOSGLUB1 | site | 656 | (+) | AACAAAC |
| ABRELATERD1 | site | 171 | (−) | ACGTG |
| ABRELATERD1 | site | 931 | (−) | ACGTG |
| ACGTABREMOTIFA2OSEM | site | 169 | (−) | ACGTGKC |
| ACGTATERD1 | site | 172 | (+) | ACGT |
| ACGTATERD1 | site | 932 | (+) | ACGT |
| ACGTATERD1 | site | 1202 | (+) | ACGT |
| ACGTATERD1 | site | 172 | (−) | ACGT |
| ACGTATERD1 | site | 932 | (−) | ACGT |
| ACGTATERD1 | site | 1202 | (−) | ACGT |
| ACGTOSGLUB1 | site | 931 | (−) | GTACGTG |
| ANAERO1CONSENSUS | site | 655 | (+) | AAACAAA |
| ANAERO1CONSENSUS | site | 334 | (−) | AAACAAA |
| ARE1 | site | 304 | (−) | RGTGACNNNGC |
| ARFAT | site | 527 | (+) | TGTCTC |
| ARFAT | site | 1213 | (+) | TGTCTC |
| ARFAT | site | 350 | (−) | TGTCTC |
| ARR1AT | site | 295 | (+) | NGATT |
| ARR1AT | site | 393 | (+) | NGATT |
| ARR1AT | site | 794 | (+) | NGATT |
| ARR1AT | site | 846 | (+) | NGATT |
| ARR1AT | site | 227 | (+) | NGATT |
| ARR1AT | site | 677 | (+) | NGATT |
| ARR1AT | site | 740 | (+) | NGATT |
| ARR1AT | site | 130 | (−) | NGATT |
| ARR1AT | site | 748 | (−) | NGATT |
| ARR1AT | site | 802 | (−) | NGATT |
| ARR1AT | site | 816 | (−) | NGATT |
| ARR1AT | site | 854 | (−) | NGATT |
| ARR1AT | site | 894 | (−) | NGATT |
| ARR1AT | site | 1271 | (−) | NGATT |
| ASF1MOTIFCAMV | site | 1363 | (+) | TGACG |
| ASF1MOTIFCAMV | site | 386 | (−) | TGACG |
| ASF1MOTIFCAMV | site | 1203 | (−) | TGACG |
| BOXCPSAS1 | site | 250 | (−) | CTCCCAC |
| BOXIINTPATPB | site | 1308 | (+) | ATAGAA |
| BOXIIPCCHS | site | 169 | (−) | ACGTGGC |
| BP5OSWX | site | 171 | (−) | CAACGTG |
| CAATBOX1 | site | 199 | (+) | CAAT |
| CAATBOX1 | site | 486 | (+) | CAAT |
| CAATBOX1 | site | 629 | (+) | CAAT |
| CAATBOX1 | site | 703 | (+) | CAAT |
| CAATBOX1 | site | 815 | (+) | CAAT |
| CAATBOX1 | site | 835 | (+) | CAAT |
| CAATBOX1 | site | 889 | (+) | CAAT |
| CAATBOX1 | site | 1102 | (+) | CAAT |
| CAATBOX1 | site | 1206 | (+) | CAAT |
| CAATBOX1 | site | 75 | (−) | CAAT |
| CAATBOX1 | site | 602 | (−) | CAAT |
| CAATBOX1 | site | 825 | (−) | CAAT |
| CAATBOX1 | site | 1013 | (−) | CAAT |
| CAATBOX1 | site | 1124 | (−) | CAAT |
| CAATBOX1 | site | 1284 | (−) | CART |
| CACTFTPPCA1 | site | 157 | (+) | YACT |
| CACTFTPPCA1 | site | 568 | (+) | YACT |
| CACTFTPPCA1 | site | 623 | (+) | YACT |
| CACTFTPPCA1 | site | 734 | (+) | YACT |
| CACTFTPPCA1 | site | 751 | (+) | YACT |
| CACTFTPPCA1 | site | 805 | (+) | YACT |
| CACTFTPPCA1 | site | 857 | (+) | YACT |
| CACTFTPPCA1 | site | 1037 | (+) | YACT |
| CACTFTPPCA1 | site | 1134 | (+) | YACT |
| CACTFTPPCA1 | site | 1244 | (+) | YACT |
| CACTFTPPCA1 | site | 26 | (+) | YACT |
| CACTFTPPCA1 | site | 342 | (+) | YACT |
| CACTFTPPCA1 | site | 557 | (+) | YACT |
| CACTFTPPCA1 | site | 561 | (+) | YACT |
| CACTFTPPCA1 | site | 935 | (+) | YACT |
| CACTFTPPCA1 | site | 1209 | (+) | YACT |
| CACTFTPPCA1 | site | 450 | (−) | YACT |
| CACTFTPPCA1 | site | 465 | (−) | YACT |
| CACTFTPPCA1 | site | 575 | (−) | YACT |
| CACTFTPPCA1 | site | 709 | (−) | YACT |
| CACTFTPPCA1 | site | 730 | (−) | YACT |
| CACTFTPPCA1 | site | 915 | (−) | YACT |
| CACTFTPPCA1 | site | 920 | (−) | YACT |
| CACTFTPPCA1 | site | 927 | (−) | YACT |
| CACTFTPPCA1 | site | 978 | (−) | YACT |
| CACTFTPPCA1 | site | 1042 | (−) | YACT |
| CACTFTPPCA1 | site | 1174 | (−) | YACT |
| CACTFTPPCA1 | site | 1359 | (−) | YACT |
| CANBNNAPA | site | 145 | (+) | CNAACAC |
| CAREOSREP1 | site | 10 | (+) | CAACTC |
| CARGCW8GAT | site | 91 | (+) | CWWWWWWWWG |
| CARGCW8GAT | site | 1278 | (+) | CWWWWWWWWG |
| CARGCW8GAT | site | 91 | (−) | CWWWWWWWWG |
| CARGCW8GAT | site | 1278 | (−) | CWWWWWWWWG |
| CATATGGMSAUR | site | 1074 | (+) | CATATG |
| CATATGGMSAUR | site | 1074 | (−) | CATATG |
| CCAATBOX1 | site | 814 | (+) | CCAAT |
| CCAATBOX1 | site | 825 | (−) | CCAAT |
| CIACADIANLELHC | site | 939 | (+) | CAANNNNATC |
| DOFCOREZM | site | 365 | (+) | AAAG |
| DOFCOREZM | site | 463 | (+) | AAAG |
| DOFCOREZM | site | 477 | (+) | AAAG |
| DOFCOREZM | site | 664 | (+) | AAAG |
| DOFCOREZM | site | 728 | (+) | AAAG |
| DOFCOREZM | site | 918 | (+) | AAAG |
| DOFCOREZM | site | 1301 | (+) | AAAG |
| DOFCOREZM | site | 1333 | (+) | AAAG |
| DOFCOREZM | site | 102 | (−) | AAAG |
| DOFCOREZM | site | 344 | (−) | AAAG |
| DOFCOREZM | site | 369 | (−) | AAAG |
| DOFCOREZM | site | 471 | (−) | AAAG |
| DOFCOREZM | site | 563 | (−) | AAAG |
| DOFCOREZM | site | 1086 | (−) | AAAG |
| DOFCOREZM | site | 1128 | (−) | AAAG |
| DOFCOREZM | site | 1238 | (−) | AAAG |
| DOFCOREZM | site | 1246 | (−) | AAAG |
| DPBFCOREDCDC3 | site | 1156 | (+) | ACACNNG |
| DPBFCOREDCDC3 | site | 691 | (−) | ACACNNG |
| DPBFCOREDCDC3 | site | 1136 | (−) | ACACNNG |
| E2FCONSENSUS | site | 1223 | (−) | WTTSSCSS |
| EBOXBNNAPA | site | 15 | (+) | CANNTG |
| EBOXBNNAPA | site | 277 | (+) | CANNTG |
| EBOXBNNAPA | site | 691 | (+) | CANNTG |
| EBOXBNNAPA | site | 1074 | (+) | CANNTG |
| EBOXBNNAPA | site | 1117 | (+) | CANNTG |
| EBOXBNNAPA | site | 1134 | (+) | CANNTG |
| EBOXBNNAPA | site | 1227 | (+) | CANNTG |
| EBOXBNNAPA | site | 15 | (−) | CANNTG |
| EBOXBNNAPA | site | 277 | (−) | CANNTG |
| EBOXBNNAPA | site | 691 | (−) | CANNTG |
| EBOXBNNAPA | site | 1074 | (−) | CANNTG |
| EBOXBNNAPA | site | 1117 | (−) | CANNTG |
| EBOXBNNAPA | site | 1134 | (−) | CANNTG |
| EBOXBNNAPA | site | 1227 | (−) | CANNTG |
| ELRECOREPCRP1 | site | 611 | (+) | TTGACC |
| EMHVCHORD | site | 562 | (−) | TGTAAAGT |
| GATABOX | site | 672 | (+) | GATA |
| GATABOX | site | 1056 | (+) | GATA |
| GATABOX | site | 1287 | (+) | GATA |
| GATABOX | site | 1369 | (+) | GATA |
| GATABOX | site | 620 | (−) | GATA |
| GATABOX | site | 945 | (−) | GATA |
| GATABOX | site | 955 | (−) | GATA |
| GATABOX | site | 1220 | (−) | GATA |
| GCCCORE | site | 264 | (+) | GCCGCC |
| GT1CONSENSUS | site | 605 | (+) | GRWAAW |
| GT1CONSENSUS | site | 297 | (−) | GRWAAW |
| GT1CONSENSUS | site | 875 | (−) | GRWAAW |
| GT1CONSENSUS | site | 1129 | (−) | GRWAAW |
| GT1CONSENSUS | site | 1239 | (−) | GRWAAW |
| GT1CORE | site | 1348 | (−) | GGTTAA |
| GT1GMSCAM4 | site | 1129 | (−) | GAAAAA |
| GTGANTG10 | site | 1362 | (+) | GTGA |
| GTGANTG10 | site | 310 | (−) | GTGA |
| GTGANTG10 | site | 388 | (−) | GTGA |
| GTGANTG10 | site | 591 | (−) | GTGA |
| GTGANTG10 | site | 622 | (−) | GTGA |
| GTGANTG10 | site | 750 | (−) | GTGA |
| GTGANTG10 | site | 804 | (−) | GTGA |
| GTGANTG10 | site | 856 | (−) | GTGA |
| GTGANTG10 | site | 1133 | (−) | GTGA |
| HEXMOTIFTAH3H4 | site | 1202 | (+) | ACGTCA |
| MARTBOX | site | 648 | (−) | TTWTWTTWTT |
| MYB1AT | site | 432 | (+) | WAACCA |
| MYB1AT | site | 238 | (+) | WAACCA |
| MYB1AT | site | 1194 | (−) | WAACCA |
| MYB2AT | site | 33 | (−) | TAACTG |
| MYB2CONSENSUSAT | site | 33 | (−) | YAACKG |
| MYB2CONSENSUSAT | site | 214 | (−) | YAACKG |
| MYBATRD22 | site | 237 | (+) | CTAACCA |
| MYBCORE | site | 33 | (+) | CNGTTR |
| MYBCORE | site | 214 | (+) | CNGTTR |
| MYBCORE | site | 221 | (+) | CNGTTR |
| MYBCORE | site | 1049 | (−) | CNGTTR |
| MYBPZM | site | 810 | (+) | CCWACC |
| MYBST1 | site | 671 | (+) | GGATA |
| MYBST1 | site | 1220 | (−) | GGATA |
| MYCATERD1 | site | 691 | (+) | CATGTG |
| MYCATRD22 | site | 691 | (−) | CACATG |
| MYCCONSENSUSAT | site | 15 | (+) | CANNTG |
| MYCCONSENSUSAT | site | 277 | (+) | CANNTG |
| MYCCONSENSUSAT | site | 691 | (+) | CANNTG |
| MYCCONSENSUSAT | site | 1074 | (+) | CANNTG |
| MYCCONSENSUSAT | site | 1117 | (+) | CANNTG |
| MYCCONSENSUSAT | site | 1134 | (+) | CANNTG |
| MYCCONSENSUSAT | site | 1227 | (+) | CANNTG |
| MYCCONSENSUSAT | site | 15 | (−) | CANNTG |
| MYCCONSENSUSAT | site | 277 | (−) | CANNTG |
| MYCCONSENSUSAT | site | 691 | (−) | CANNTG |
| MYCCONSENSUSAT | site | 1074 | (−) | CANNTG |
| MYCCONSENSUSAT | site | 1117 | (−) | CANNTG |
| MYCCONSENSUSAT | site | 1134 | (−) | CANNTG |
| MYCCONSENSUSAT | site | 1227 | (−) | CANNTG |
| NAPINMOTIFBN | site | 692 | (−) | TACACAT |
| NODCON2GM | site | 188 | (+) | CTCTT |
| NODCON2GM | site | 1334 | (−) | CTCTT |
| NODCON2GM | site | 1356 | (−) | CTCTT |
| NTBBF1ARROLB | site | 562 | (+) | ACTTTA |
| NTBBF1ARROLB | site | 462 | (−) | ACTTTA |
| NTBBF1ARROLB | site | 917 | (−) | ACTTTA |
| OSE2ROOTNODULE | site | 188 | (+) | CTCTT |
| OSE2ROOTNODULE | site | 1334 | (−) | CTCTT |
| OSE2ROOTNODULE | site | 1356 | (−) | CTCTT |
| P1BS | site | 84 | (+) | GNATATNC |
| P1BS | site | 84 | (−) | GNATATNC |
| POLASIG1 | site | 651 | (+) | AATAAA |
| POLASIG1 | site | 992 | (+) | AATAAA |
| POLASIG1 | site | 376 | (−) | AATAAA |
| POLASIG1 | site | 599 | (−) | AATAAA |
| POLASIG2 | site | 371 | (−) | AATTAAA |
| POLASIG3 | site | 648 | (+) | AATAAT |
| POLLEN1LELAT52 | site | 104 | (−) | AGAAA |
| POLLEN1LELAT52 | site | 194 | (−) | AGAAA |
| POLLEN1LELAT52 | site | 770 | (−) | AGAAA |
| PREATPRODH | site | 12 | (+) | ACTCAT |
| PREATPRODH | site | 357 | (+) | ACTCAT |
| PREATPRODH | site | 912 | (−) | ACTCAT |
| PROLAMINBOXOSGLUB1 | site | 474 | (+) | TGCAAAG |
| PROLAMINBOXOSGLUB1 | site | 471 | (−) | TGCAAAG |
| PYRIMIDINEBOXOSRAMY1A | site | 101 | (+) | CCTTTT |
| PYRIMIDINEBOXOSRAMY1A | site | 1085 | (+) | CCTTTT |
| QELEMENTZMZM13 | site | 612 | (−) | AGGTCA |
| RAV1AAT | site | 1049 | (+) | CAACA |
| RAV1AAT | site | 77 | (−) | CAACA |
| RAV1AAT | site | 1249 | (−) | CAACA |
| REALPHALGLHCB21 | site | 433 | (+) | AACCAA |
| REALPHALGLHCB21 | site | 812 | (+) | AACCAA |
| REBETALGLHCB21 | site | 1220 | (−) | CGGATA |
| ROOTMOTIFTAPDX1 | site | 73 | (+) | ATATT |
| ROOTMOTIFTAPDX1 | site | 86 | (+) | ATATT |
| ROOTMOTIFTAPDX1 | site | 1149 | (−) | ATATT |
| RYREPEATBNNAPA | site | 137 | (+) | CATGCA |
| RYREPEATLEGUMINBOX | site | 137 | (+) | CATGCAY |
| S1FBOXSORPS1L21 | site | 45 | (−) | ATGGTA |
| S1FBOXSORPS1L21 | site | 69 | (−) | ATGGTA |
| S1FBOXSORPS1L21 | site | 111 | (−) | ATGGTA |
| S1FBOXSORPS1L21 | site | 980 | (−) | ATGGTA |
| SEBECONSSTPR10A | site | 526 | (+) | YTGTCWC |
| SEBECONSSTPR10A | site | 1212 | (+) | YTGTCWC |
| SEBECONSSTPR10A | site | 350 | (−) | YTGTCWC |
| SEF4MOTIFGM7S | site | 337 | (+) | RTTTTTR |
| SITEIIATCYTC | site | 1303 | (−) | TGGGCY |
| SORLIP1AT | site | 169 | (+) | GCCAC |
| SORLIP1AT | site | 901 | (+) | GCCAC |
| SORLIP1AT | site | 273 | (−) | GCCAC |
| SV4000REENHAN | site | 1193 | (+) | GTGGWWHG |
| SV4000REENHAN | site | 237 | (−) | GTGGWWHG |
| T/GBOXATPIN2 | site | 171 | (−) | AACGTG |
| TAAAGSTKST1 | site | 364 | (+) | TAAAG |
| TAAAGSTKST1 | site | 462 | (+) | TAAAG |
| TAAAGSTKST1 | site | 663 | (+) | TAAAG |
| TAAAGSTKST1 | site | 917 | (+) | TAAAG |
| TAAAGSTKST1 | site | 1332 | (+) | TAAAG |
| TAAAGSTKST1 | site | 563 | (−) | TAAAG |
| TATABOX2 | site | 1279 | (+) | TATAAAT |
| TATABOX4 | site | 93 | (+) | TATATAA |
| TATABOX4 | site | 92 | (−) | TATATAA |
| TATABOX5 | site | 596 | (+) | TTATTT |
| TATABOX5 | site | 991 | (−) | TTATTT |
| TBOXATGAPB | site | 1245 | (+) | ACTTTG |
| TGACGTVMAMY | site | 1202 | (−) | TGACGT |
| UPRMOTIFIIAT | site | 156 | (+) | CCNNNNNNNNNNNNCCACG |
| VSF1PVGRP18 | site | 211 | (+) | GCTCCGTTG |
| WBOXATNPR1 | site | 611 | (+) | TTGAC |
| WBOXATNPR1 | site | 1204 | (−) | TTGAC |
| WBOXHVISO1 | site | 38 | (−) | TGACT |
| WBOXNTERF3 | site | 612 | (+) | TGACY |
| WBOXNTERF3 | site | 38 | (−) | TGACY |
| WBOXNTERF3 | site | 308 | (−) | TGACY |
| WBOXNTERF3 | site | 446 | (−) | TGACY |
| WRKY71OS | site | 612 | (+) | TGAC |
| WRKY71OS | site | 1002 | (+) | TGAC |
| WRKY71OS | site | 1099 | (+) | TGAC |
| WRKY71OS | site | 1363 | (+) | TGAC |
| WRKY71OS | site | 39 | (−) | TGAC |
| WRKY71OS | site | 135 | (−) | TGAC |
| WRKY71OS | site | 309 | (−) | TGAC |
| WRKY71OS | site | 387 | (−) | TGAC |
| WRKY71OS | site | 447 | (−) | TGAC |
| WRKY71OS | site | 590 | (−) | TGAC |
| WRKY71OS | site | 1204 | (−) | TGAC |

1-34. (canceled)

35. An isolated nucleic acid comprising a nucleotide sequence which corresponds to a promoter-active region of a gene comprising a transcribable DNA sequence encoding SEQ ID NO:28.

36. The isolated nucleic acid of claim 35, wherein the promoter-active region comprises a nucleotide sequence as set forth in SEQ ID NO: 1, or a variant thereof.

37. The isolated nucleic acid of claim 35, wherein the variant comprises a nucleotide sequence with at least 50% sequence identity to the nucleotide sequence as set forth in SEQ ID NO: 1.

38. The isolated nucleic acid of claim 36, wherein the variant comprises a nucleotide sequence selected from the group consisting of SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; and SEQ ID NO:15.

39. An isolated nucleic acid comprising a nucleotide sequence as set forth in SEQ ID NO: 1, or a variant thereof.

40. The isolated nucleic acid of claim 39, wherein the variant comprises a nucleotide sequence selected from the group consisting of SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; and SEQ ID NO:15.

41. The isolated nucleic acid of claim 39, wherein the variant has at least 50% sequence identity to the nucleotide sequence as set forth in SEQ ID NO: 1.

42. The isolated nucleic acid of claim 39, which comprises a biologically-active fragment of a nucleotide sequence as set forth in SEQ ID NO: 1, or a variant thereof.

43. The isolated nucleic acid of claim 42, wherein the biologically-active fragment is a promoter-active fragment.

44. An isolated gene comprising the isolated nucleic acid of claim 35 operably linked to a transcribable DNA sequence encoding SEQ ID NO:28.

45. A chimeric gene comprising the isolated nucleic acid of claim 35, operably linked to a heterologous nucleic acid.

46. The chimeric gene of claim 45, wherein the heterologous nucleic acid encodes a biologically-active protein.

47. A genetic construct comprising the isolated nucleic acid of claim 35, together with one or more other nucleotide sequences.

48. The genetic construct of claim 47, wherein the one or more other nucleotide sequences is selected from the group consisting of an enhancer of transcription, an enhancer of translation, a nucleotide sequence for autonomous replication in a prokaryote, a regulatory element for mRNA processing, a selectable marker and a screenable marker.

49. The genetic construct of claim 47, which is an expression vector.

50. The genetic construct of claim 49, wherein the expression vector comprises the isolated nucleic acid of claim 1.

51. The genetic construct of claim 47, which is an expression construct.

52. An expression vector comprising the isolated nucleic acid of a promoter-active region of a gene comprising a transcribable DNA sequence encoding SEQ ID NO:28 operably linked to a heterologous nucleic acid.

53. The genetic construct of claim 47 characterized in that said isolated nucleic acid is capable of directing transcription in the endosperm of wheat.

54. A host cell comprising the genetic construct of claim 47.

55. The host cell of claim 54, which is derived from a plant.

56. The host cell of claim 55, wherein the plant is a cereal.

57. The host cell of claim 56, wherein the cereal is wheat.

58. A method of producing a recombinant protein, said method including the step of introducing into a plant host cell or tissue the genetic construct of claim 47, wherein said genetic construct is capable of producing said recombinant protein.

59. The method of claim 58, wherein the plant host cell or tissue is derived from a cereal.

60. The method of claim 59, wherein the cereal is wheat.

61. A method of facilitating targeted expression to a plant endosperm, wherein said method includes the step of expressing the chimeric gene of claim 45 in the endosperm of a plant.

62. The method of claim 61, wherein the plant is a cereal.

63. The method of claim 62, wherein the plant is wheat.

64. A genetically-transformed plant comprising the isolated nucleic acid of claim 35.

65. The genetically-transformed plant of claim 64, wherein the genetically-transformed plant has an altered phenotype compared to a corresponding non-transformed plant.

66. The genetically-transformed plant of claim 65, wherein the altered phenotype results from expression of a heterologous nucleic acid.

67. The genetically-transformed plant of claim 64, which is a cereal.

68. The genetically-transformed plant of claim 66, wherein the cereal is wheat.

69. An isolated nucleic acid encoding an isolated protein comprising an amino acid sequence as set forth in SEQ ID NO:28.

70. An isolated polypeptide encoded by the isolated nucleic acid of claim 69.

71. An antibody, or a fragment thereof, which binds to the isolated polypeptide of claim 70, or a fragment thereof.
